(19) United States
(12) Patent Application Publication
(10) Pub. No.: US 2010/0028867 A1
(43) Pub. Date: Feb. 4, 2010

(54) LRRTM1 Compositions and Methods of Their Use for the Diagnosis and Treatment of Cancer

(76) Inventors: Elizabeth Bosch, Cupertino, CA (US); Kevin Hestir, Kensington, CA (US); Justin Wong, Oakland, CA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/084,172

(22) PCT Filed: Oct. 25, 2006
(86) PCT No.: PCT/US2006/041852

§ 371 Date: Jun. 2, 2009

Related U.S. Application Data
(60) Provisional application No. 60/730,652, filed on Oct. 26, 2005.

(51) Int. Cl. C12Q 1/68; G01N 33/574

(52) U.S. Cl. 435/6; 435/7.23

(57) ABSTRACT

Microarray analysis, confirmed by RT-PCR, demonstrated that mRNA from certain cancer tissues, for example, ovarian cancer tissue, pancreatic cancer tissue, and colorectal cancer tissue, hybridizes specifically and preferentially to LRRTM1. LRRTM1 is a leucine-rich repeat transmembrane protein overexpressed on the surface of cancer cells compared to normal tissues and thus provides a therapeutic target for treating cancer. Modulators of LRRTM1, highly expressed in cancerous as compared to normal tissues, are provided for the diagnosis and treatment of proliferative disorders such as cancer. The invention further provides methods of treating cancer with therapeutic agents directed toward LRRTM1.

### PRIORITY CLAIM

This application claims the benefit of provisional application 60/730,652, filed in the United States Patent and Trademark Office on Oct. 26, 2005, the disclosure of which is hereby incorporated by reference.

### TECHNICAL FIELD

This invention relates to human LRRTM1 polynucleotides and their encoded polypeptides which are highly expressed in cancer tissues, for example, ovarian, pancreatic, and colon/colorectal cancer tissues. The invention also relates to modulators, for example, antibodies, of such polynucleotides and polypeptides that specifically bind to and/or interfere with the activity of these polypeptides, polynucleotides, their fragments, variants, and antagonists. The invention further relates to compositions containing such polypeptides, polynucleotides, or modulators thereof, and uses of such compositions in methods of treating proliferative disorders, including cancer. The invention additionally relates to methods of diagnosing proliferative disorders, such as cancer, by detecting these polynucleotides, polypeptides, or antibodies thereto in body fluid samples. The invention provides diagnostic tests which identify LRRTM1 polypeptides and polynucleotides that correlate with particular disorders.

### BACKGROUND ART

The American Cancer Society estimates that approximately 1,400,000 new cases of cancer will have been diagnosed in the United States in 2004, and that approximately 570,000 cancer patients will have died of the disease. An estimated 26,000 of these new cases will be diagnosed as ovarian cancer, and an estimated 16,000 patients will have died of ovarian cancer in 2004. In the 19% of cases where the cancer is detected early, before it has spread beyond the ovaries, the five-year survival rate is 90%. However, because most ovarian cancers are not diagnosed at such an early stage, 50% of the women currently diagnosed with ovarian cancer die from it within five years. Ovarian cancer may be difficult to diagnose because the symptoms can be confused with other diseases, and because there is no reliable, easy-to-administer screening test. See American Cancer Society (2005) Cancer Facts & Figures (available at http://www.cancer.org/docroot/STT/stt_{—}0.asp); About Ovarian Cancer (available at http://www.ovariancancer.org/content/1-5-1.html).

An estimated 32,180 of the newly diagnosed cancers in 2005 will be diagnosed as cancer of the pancreas, with that number about evenly divided between men and women. An estimated 31,800 patients will die of pancreatic cancer in 2005, again with that number about evenly divided between men and women. Pancreatic cancer is the fourth leading cause of cancer death overall with only about 23% of patients alive one year after diagnosis, and only about 4% alive five years after diagnosis. Even patients diagnosed with local disease, indicating that the cancer has not spread to other organs, have only a five-year relative survival rate of 15%. See American Cancer Society (2005), What are the Key Statistics About Cancer of the Pancreas? (available at http://www.cancer.org/docroot/CRI/content/CRI_{—}2_{—}4_{—}1X_What_are_the_key_statistics_for_pancreatic_cancer_{—}34.asp?sitearea=).

An estimated 145,000 of the newly diagnosed cancers in 2005 will be diagnosed as cancer of the colon or rectum (colorectal cancer) and an estimated 56,000 patients will have died of this disease. In its early stages, colorectal cancer usually causes no symptoms. When it is detected at an early, localized stage, the five year survival rate is 90%. However, only 39% of colorectal cancers are discovered at this stage. See American Cancer Society, Colorectal Cancer Facts & FIGS., Special Edition 2005, (available at http://www.cancer.org/docroot/STT/stt_{—}0.asp). Therefore, diagnostic markers for ovarian cancer, including early stage ovarian cancer, pancreatic cancer, and colorectal cancer, will have a significant impact on cancer morbidity and mortality.

The main treatment options for cancer, including ovarian, pancreatic, and colorectal cancer, include surgery, chemotherapy, and radiation therapy, none of which are universally effective at treating the cancer and each of which is associated with undesirable risks and side effects. For colorectal cancer, newer therapeutic drugs based on monoclonal antibodies have begun to be used clinically. These include Cetuximab (Erbitux®) and Bevacizumab (Avastin®). Cetuximab is an antibody against the epidermal growth factor receptor (EGFR) protein, which is overexpressed in some tumor cells. Cetuximab is used in combination with chemotherapy to treat advanced colorectal cancer. Bevacizumab is an antibody against the vascular endothelial growth factor (VEGF) protein. Bevacizumab inhibits angiogenesis and is used in combination with chemotherapy to treat metastatic colorectal cancer. Therefore, while promising for a subset of patients, Cetuximab and Bevacizumab are not useful against all types of colorectal cancer. See Cancer Reference Information (available at http://www.cancer.org/docroot/CRI/CRI_{—}0.asp). Accordingly, new therapies are needed for the treatment of cancer, for example, ovarian, pancreatic, and colorectal cancers.

The LRRTM (leucine-rich repeat transmembrane neuronal) family of proteins comprise a protein module known as a leucine-rich repeat (LRR). LRRs are repetitive sequences present in a number of proteins with diverse functions. They are commonly involved in protein-protein interactions and are found in the extracellular region of transmembrane proteins and in secreted proteins involved in ligand-receptor interactions or in cell adhesion. LRRs are generally about 20-29 amino acids in length. (Kobe, B. and Kajava, A. V. (2001) Curr. Opin. Struct. Biol. 11:725-32). They may be present in tandem fashion and comprise a consensus sequence of LxxLxxLxLxxNxLxxLxxxxFxx, where L corresponds to leucine, valine, isoleucine, or phenylalanine and x corresponds to any amino acid. (Kajava, A. V. (1998) J. Mol. Biol. 277:519-527). Analysis of the crystal structures of several LRR-containing proteins reveal that LRR forms a slightly curved chain with parallel P sheets on the concave side and mostly helical structures on the opposite side. It is believed that molecular interactions occur with individual amino acids on the concave side of the LRRs. The LRRs are flanked by cysteine-rich N- and C-terminal flanking regions that prevent the hydrophobic core of the LRRs from being exposed to solvent. (Lauren, J. et al., (2003) Genomics 81:411-421).

The genes encoding LRRTM proteins were first identified in human and mouse and have subsequently been found in other vertebrate species, but not in invertebrates. (Lauren et al. at 416). In human, as well as mouse, there are four LRRTM genes (LRRTM1-4) Id. at 411-12. The LRRTM1 gene is located on chromosome 2p12 within an intron of α2-catenin, which mediates the adhesive properties of the homophilic adhesion molecules cadherins. Id. at 412.

LRRTM1 protein can be expressed as a 522 amino acid transmembrane protein with an extracellular domain comprising 10 LRRs, a transmembrane domain, and a cytoplasmic domain; it is designated NP_{—}849161.1 in the National Center for Biotechnology Information (NCBI) database. (Clark et al., (2003) Genome Res. 13:2265-70). The corresponding nucleic acid sequence is designated NM_{—}178839.3 in the NCBI database. Id. When normal human tissues were analyzed, LRRTM1 mRNA was found to be highly expressed in brain, mainly cortical neurons, and salivary gland of normal human tissue. Intermediate levels of expression were detected in the cerebellum, small intestine, spinal cord, stomach, testis, and uterus. Expression in the colon was undetectable. (Lauren et al. at 414, 416, 418.) LRRTM1 is implicated in embryonic brain development and maintenance because LRRTM1 mRNA expression is detected as early as embryonic day 13 and continues to increase throughout prenatal development. (Id. at 415, 416.) In addition, the intracellular regions of all LRRTM proteins, including LRRTM1, contain several conserved tyrosine, serine, and threonine residues that may be phosphorylated and thus, may be involved in signal transduction. Moreover, the C-termini of all LRRTM proteins have the amino acid sequence “glutamic acid-cysteine-glutamic acid-valine (ECEV).” (SEQ ID NO: 57) (Id. at 414.)

The invention provides LRRTM1 compositions and methods for using LRRTM1 to improve diagnostic, prognostic, and treatment procedures for proliferative diseases, such as cancer. The invention also provides modulators of LRRTM1 with acceptable safety profiles that can be used in the diagnosis and treatment of proliferative diseases. These modulators may be, for example, antibodies. The invention can be embodied, for example, as follows.

### SUMMARY

The invention provides methods of diagnosing cancer in a subject, or assigning a prognostic risk of one or more future clinical outcomes to a subject suffering from cancer, comprising performing an assay configured to detect a soluble form of LRRTM1 in a body fluid sample obtained from the subject; obtaining a result from the assay; and relating the result of the assay to the presence or absence of cancer in the subject, or to the prognostic risk of one or more clinical outcomes for the subject. In certain embodiments, the cancer is ovarian cancer, pancreatic cancer, or colon cancer. In certain embodiments, the body fluid sample is obtained from blood, serum, or plasma. In an embodiment, the soluble form of LRRTM1 comprises all or a portion of the extracellular domain of full length LRRTM1, the full length LRRTM1 having the sequence depicted in SEQ. ID. NO.:26.

In certain embodiments, performing the assay comprises contacting the body fluid sample with an antibody that binds to the extracellular domain of full length LRRTM1, and detecting polypeptide binding to the antibody. In an embodiment, the assay is a sandwich immunoassay.

In an embodiment, the assay result is expressed as an amount of the soluble form of LRRTM1, and the “relating” step comprises comparing the assay result to a predetermined threshold level of the soluble form of LRRTM1, and performing one or more of the following determinations: diagnosing the presence of cancer in the subject if the assay result is greater than the threshold level; diagnosing the absence of cancer in the subject if the assay result is less than the threshold level; assigning an increased likelihood of a poor prognostic outcome if the assay result is greater than the threshold level relative to the prognostic risk assigned; or assigning a decreased likelihood of a poor prognostic outcome if the assay result is less than the threshold level relative to the prognostic risk assigned.

The invention also provides methods of determining the presence of a polypeptide specifically binding to an antibody in a sample, comprising allowing an antibody to interact with the sample and determining whether interaction between the antibody and the polypeptide has occurred, wherein the antibody specifically recognizes, binds to, interferes with, and/or modulates the biological activity of at least one polypeptide and/or polynucleotide a biologically active fragment thereof, as shown in the Tables, Sequence Listing, or Figures, wherein the polypeptide is other than a full-length LRRTM1 of SEQ. ID. NO.:26.

The invention further provides methods of determining the presence of an antibody specifically binding to a polypeptide or a polynucleotide in a sample, comprising allowing an isolated polynucleotide encoding a polypeptide or an isolated polypeptide, wherein the polypeptide comprises an amino acid sequence as shown in the Tables, Figures, or Sequence Listing, or biologically active fragments thereof, to interact with the sample; and determining whether interaction between the antibody and the polypeptide or polynucleotide has occurred.

The invention yet further provides methods of diagnosing cancer in a subject, comprising providing an antibody; allowing the antibody to contact a body fluid sample; and detecting specific binding between the antibody and an antigen in the sample to determine whether the subject has cancer, wherein the antibody specifically binds to at least one polypeptide and/or polynucleotide as shown in the Tables, Sequence Listing, or Figures, or a biologically active fragment thereof, wherein the polypeptide is other than a full-length LRRTM1 of SEQ. ID. NO.:26. In certain embodiments, the cancer is chosen from ovarian cancer, pancreatic cancer, and colorectal cancer.

In addition, the invention provides methods of diagnosing cancer in a subject, comprising providing a polypeptide that specifically binds to an antibody allowing the polypeptide to contact a body fluid sample; and detecting specific binding between the polypeptide and any interacting molecule in the sample to determine whether the subject has cancer, wherein the antibody specifically binds to at least one polypeptide and/or polynucleotide as shown in the Tables, Sequence Listing, or Figures or a biologically active fragment thereof, wherein the polypeptide is other than a full-length LRRTM1 of SEQ. ID. NO.:26. In certain embodiments, the cancer is chosen from ovarian cancer, pancreatic cancer, and colorectal cancer.

In certain embodiments, the invention provides kits comprising an antibody as described herein and instructions for performing such methods. In certain such embodiments, the antibody specifically recognizes, binds to, interferes with, and/or modulates the biological activity of at least one polypeptide and/or polynucleotide or a biologically active fragment thereof, as shown in the Tables, Sequence Listing, or Figures, wherein the polypeptide is other than a full-length LRRTM1 of SEQ. ID. NO.:26.

In another aspect, the invention provides an isolated polynucleotide encoding a polypeptide, wherein the polypeptide comprises an amino acid sequence as shown in the Tables, Figures, or Sequence Listing, or a biologically active fragment thereof, wherein the polypeptide is other than a full-length LRRTM1 of SEQ. ID. NO.:26. In an embodiment, the polynucleotide is chosen from a RNAi molecule, a ribozyme, and a nucleotide aptamer.

The invention also provides an isolated polynucleotide, wherein the polynucleotide is a complement of a polynucleotide encoding a polypeptide, wherein the polypeptide comprises an amino acid sequence as shown in the Tables, Figures, or Sequence Listing, or a biologically active fragment thereof, wherein the polypeptide is other than a full-length LRRTM1 of SEQ. ID. NO.:26. In an embodiment, this polynucleotide is chosen from a RNAi molecule, a ribozyme, and a nucleotide aptamer.

The invention further provides an isolated polypeptide encoded by a polynucleotide, wherein the polypeptide comprises an amino acid sequence as shown in the Tables, Figures, or Sequence Listing, or a biologically active fragment thereof, wherein the polypeptide is other than a full-length LRRTM1 of SEQ. ID. NO.:26.

The invention yet further provides fusion molecules comprising a first polypeptide, wherein the first polypeptide comprises an amino acid sequence of any of the above-described polypeptides; and a fusion partner, wherein the fusion partner is chosen from a polymer, a polypeptide, Fc region, human serum albumin, a part of human serum albumin, albumin, fetuin A, fetuin B, a leucine zipper domain, a tetranectin trimerization domain, a mannose binding protein, for example, mannose binding protein 1, and an immunoglobulin constant domain. In certain embodiments, fusion molecules further comprise a secretion signal sequence. In certain embodiments, the invention provides polynucleotides encoding fusion molecules. In an embodiment, the fusion molecule has a higher plasma stability than the first polypeptide absent the fusion partner. In an embodiment, the fusion molecule further comprises a linker. In a further embodiment, the linker is a peptide linker.

In yet another aspect, the invention provides pharmaceutical compositions comprising a pharmaceutically acceptable carrier and an isolated polynucleotide encoding any of the above-described polypeptides; or a complement thereof.

The invention also provides pharmaceutical compositions comprising a pharmaceutically acceptable carrier and any of the above-described isolated polypeptides encoded by any of the above polynucleotides.

The invention further provides non-human animals injected with any of the above-described isolated polynucleotides encoding a polypeptide; or a complement thereof.

The invention yet further provides non-human animals injected with any of the above-described isolated polypeptides encoded by a polynucleotide.

The invention provides isolated antibodies specifically recognizing, binding to, interfering with, and/or modulating the biological activity of at least one polypeptide, polynucleotide, and/or biologically active fragment thereof, as shown in the Tables, Sequence Listing, or Figures, wherein the polypeptide is other than a full-length LRRTM1 of SEQ. ID. NO.:26. In an embodiment, the polypeptide comprises a fragment chosen from Table 1, wherein the fragment is an N-terminal fragment, a leucine-rich repeat fragment, and/or a C-terminal fragment.

In an embodiment, the antibody further comprises one or more cytotoxic component chosen from a radioisotope, a microbial toxin, a plant toxin, and a chemical compound. In an embodiment, the antibody has a function chosen from specifically inhibiting the binding of the polypeptide to a ligand, specifically inhibiting the binding of the polypeptide to a substrate, specifically inhibiting the binding of the polypeptide as a ligand, specifically inhibiting the binding of the polypeptide as a substrate, inducing antibody-dependent cell cytotoxicity, inducing complement-dependent cytotoxicity, modulating ligand/receptor interaction, and modulating enzyme/substrate interaction.

In certain embodiments, the antibody is chosen from one or more of a monoclonal antibody; a polyclonal antibody; a single chain antibody; an antibody comprising a backbone of a molecule with an Ig domain or a T cell receptor backbone; a targeting antibody; a neutralizing antibody; a stabilizing antibody; an enhancing antibody; an antibody agonist; an antibody antagonist; an antibody that promotes endocytosis of a target antigen; a cytotoxic antibody, an antibody that mediates antibody-dependent cell cytotoxicity, an antibody that mediates complement-dependent cytotoxicity; a human antibody, a non-human primate antibody; a non-primate animal antibody; a rabbit antibody, a mouse antibody; a rat antibody; a sheep antibody; a goat antibody; a horse antibody; a porcine antibody; a cow antibody, a chicken antibody; a humanized antibody; a primatized antibody; a chimeric antibody; an antigen binding fragment; a fragment comprising a variable region of a heavy chain or a light chain of an immunoglobulin; a fragment comprising a complementarity determining region or a framework region of an immunoglobulin; and one or more active fragments, analogues, and/or antagonists of one or more of these antibodies. In an embodiment, the antibody is a monoclonal antibody. In an embodiment, the antibody is an antigen-binding fragment of an immunoglobulin.

The invention also provides a pharmaceutical composition comprising a pharmaceutically acceptable carrier and any of the above-described antibodies.

In certain embodiments, the antibody is produced in a plant, an animal, or a cell. In certain such embodiments, the invention provides a host cell genetically modified to produce any of the above-described antibodies. In an embodiment, the cell is chosen from a bacterial cell, a fungal cell, a plant cell, an insect cell, and a mammalian cell. In an embodiment, the cell is chosen from a yeast cell, an Aspergillus cell, an SF9 cell, a High Five cell, a cereal plant cell, a tobacco cell, a tomato cell, a 293 cell, a myeloma cell, a NS0 cell, a PerC6 cell, and a CHO cell.

The invention further provides an epitope of LRRTM1, wherein the epitope is chosen from HNLSGLLGLSLRYNSLSELRAGQF (SEQ. ID. NO. 14), TGLMQLT WLYLDHNHICSVQGDAF (SEQ. ID. NO. 15), QKLRRVKELTLSSNQITQLPNTTF (SEQ. ID. NO. 16), RPMPNLRSVDLSYNKLQALAPDLF (SEQ. ID. NO. 17), HGLRKLTTLHMRANAIQFVPVRIF (SEQ. ID. NO. 18), QDCRSLKFLDIGYNQ LKSLARNSF (SEQ. ID. NO. 19), AGLFKLTELHLEHNDLVKVNFAHF (SEQ. ID. NO. 20), PRLISLHSLCLRRNKVAUVVSSLD (SEQ. ID. NO. 21), DWVWNLE KMDLSGNEIEYMEPHVF (SEQ. ID. NO. 22), ETVPHLQSLQLDSNRLTYIEPRIL (SEQ. ID. NO. 23), AAPSGCPQLCRCEGRLLYCEALNLT (SEQ. ID. NO. 24), and LTSITLAGNLWDCGRNVCALASWLNNFQGRYDGNLQCASPE (SEQ. ID. NO. 25).

The invention yet further provides a bacteriophage displaying any of the above-described antibodies and/or fragments thereof.

In another aspect, the invention provides methods of modulating a biological activity of a first human or non-human animal cell comprising providing any of the above-described antibodies; and contacting the antibody with the first cell, wherein the activity of the first cell, and/or a second cell, is modulated. In an embodiment, the modulation of biological activity is chosen from enhancing cell activity directly, enhancing cell activity indirectly, inhibiting cell activity directly, inhibiting cell activity indirectly, inducing antibody-dependent cell cytotoxicity, and inducing complement-dependent cytotoxicity.

In an embodiment, the modulated cell activity is chosen from signal transduction, transcription, and translation. In an embodiment, the modulation of cell activity results in cell death and/or inhibition of cell growth.

In certain embodiments, contacting the antibody with a first cell results in recruitment of at least one second cell. In certain such embodiments, the first cell is a cancer cell. In an embodiment, the first or second cell is chosen from a T cell, B cell, NK cell, dendritic cell, antigen presenting cell, and macrophage.

In yet another aspect, the invention provides methods of identifying a modulator of the biological activity of a polypeptide comprising providing at least one polypeptide as shown in the sequences listed in the Tables, Figures, or Sequence Listing, or biologically active fragments thereof; allowing at least one agent to contact the polypeptide; and selecting an agent that binds the polypeptide or affects the biological activity of the polypeptide. In an embodiment, the modulator is an antibody or a fragment thereof.

The invention also provides a modulator composition comprising a pharmaceutically acceptable carrier and a modulator, wherein the modulator is obtainable by the above-described methods.

The invention further provides a modulator composition comprising a pharmaceutically acceptable carrier and a modulator, wherein the modulator is any of the above-described antibodies.

The invention yet further provides a modulator composition comprising a pharmaceutically acceptable carrier and a modulator, wherein the modulator is a soluble receptor that competes for ligand binding to an isolated polypeptide comprising an amino acid sequence as shown in the Tables, Figures, or Sequence Listing, or biologically active fragments thereof.

In addition, the invention provides a modulator composition comprising a pharmaceutically acceptable carrier and a modulator, wherein the modulator is an extracellular fragment that competes for ligand binding to an isolated polypeptide comprising an amino acid sequence as shown in the Tables, Figures, or Sequence Listing, or biologically active fragments thereof.

The invention also provides a modulator composition comprising a pharmaceutically acceptable carrier and a modulator, wherein the modulator comprises an RNAi molecule, a ribozyme, or an antisense molecule, wherein the modulator inhibits the transcription or translation of an isolated polynucleotide or an isolated polypeptide comprising an amino acid sequence encoded by a polynucleotide as shown in the Tables, Figures, or Sequence Listing, or biologically active fragments thereof.

The invention further provides a modulator composition comprising a pharmaceutically acceptable carrier and a modulator, wherein the modulator is an aptamer that inhibits the function of an isolated polynucleotide or an isolated polypeptide comprising an amino acid sequence encoded by a polynucleotide as shown in the Tables, Figures, or Sequence Listing, or biologically active fragments thereof.

In another aspect, the invention provides methods of treating uncontrolled proliferative growth in a subject comprising administering a modulator which binds to or interferes with the activity of an isolated polynucleotide encoding a polypeptide or an isolated polypeptide encoded by a polynucleotide, wherein the polypeptide comprises an amino acid sequence as shown in the Tables, Figures, or Sequence Listing, or biologically active fragments thereof, to a subject. In an embodiment, the modulator is any of the above-described antibodies. In an embodiment, the uncontrolled proliferative growth is a tumor. In an embodiment, the tumor is chosen from an ovarian tumor, pancreatic tumor, and a colorectal tumor.

The invention also provides a method of treating a tumor in a subject comprising providing the above-described modulator composition and administering the modulator composition to the subject. In an embodiment, the modulator is an antibody. In certain embodiments, the antibody specifically recognizes, binds to, or modulates the biological activity of a polypeptide and wherein the polypeptide comprises an amino acid sequence as shown in the Tables, Figures, or Sequence Listing or is encoded by a polynucleotide as shown in the Tables, Figures, or Sequence Listing, or biologically active fragments thereof.

The invention further provides a method of treating an ovarian tumor in a subject comprising providing any of the above-described modulator compositions and administering the modulator composition to the subject. In certain embodiments, the modulator is an antibody. In certain of those embodiments, the antibody specifically recognizes, binds to, or modulates the biological activity of a polypeptide and wherein the polypeptide comprises an amino acid sequence as shown in the Tables, Figures, or Sequence Listing or is encoded by a polynucleotide as shown in the Tables, Figures, or Sequence Listing, or biologically active fragments thereof.

The invention yet further provides a method of treating a colorectal tumor in a subject comprising providing any of the above-described modulator compositions and administering the modulator composition to the subject. In certain embodiments, the modulator is an antibody. In certain of those embodiments, the antibody specifically recognizes, binds to, or modulates the biological activity of a polypeptide and wherein the polypeptide comprises an amino acid sequence as shown in the Tables, Figures, or Sequence Listing or is encoded by a polynucleotide as shown in the Tables, Figures, or Sequence Listing, or biologically active fragments thereof.

In addition, the invention provides a method of treating a pancreatic tumor in a subject comprising providing any of the above-described modulator compositions and administering the modulator composition to the subject. In certain embodiments, the modulator is an antibody. In certain of those embodiments, the antibody specifically recognizes, binds to, or modulates the biological activity of a polypeptide and wherein the polypeptide comprises an amino acid sequence as shown in the Tables, Figures, or Sequence Listing or is encoded by a polynucleotide as shown in the Tables, Figures, or Sequence Listing, or biologically active fragments thereof.

In another aspect, the invention provides kits comprising any of the above-described antibodies and instructions for performing any of the above-described methods.

The invention provides a method for prophylactic treatment of cancer or therapeutic treatment of cancer of a subject in need thereof, comprising providing a vaccine and administering the vaccine to the subject, wherein the vaccine comprises a polynucleotide or a polypeptide chosen from at least one sequence according to SEQ. ID. NO.:1-28 or a complement, biologically active fragment, or variant thereof. In an embodiment, the vaccine is a cancer vaccine for ovarian cancer. In an embodiment, the vaccine is a cancer vaccine for pancreatic cancer. In an embodiment, the vaccine is a cancer vaccine for colon cancer.

### INDUSTRIAL APPLICABILITY

LRRTM1 polypeptides, polynucleotides, and modulators, for example, antibodies, find use in a number of diagnostic, prophylactic, and therapeutic applications relating to proliferative disorders, for example, cancer. These therapeutics include nucleic acid and polypeptide antibodies and vaccines, such as cancer vaccines, which may be administered alone, such as naked DNA, or may be facilitated, such as via viral vectors, microsomes, liposomes, or nanoparticles. Therapeutic antibodies include, for example, monoclonal antibodies or binding fragments. They may be administered alone or in combination with cytotoxic agents, such as radioactive or chemotherapeutic agents.

### BRIEF DESCRIPTION OF THE TABLES AND FIGURES

### Brief Description of the Tables

Table 1 provides information regarding the sequences listed in the Sequence Listing. Column 1 shows an internally designated identification number (FP ID) for SEQ. ID. NOS. 1-26. Column 2 shows the nucleotide sequence ID number for the nucleic acid sequences of the Sequence Listing (SEQ. ID. NO. (N1)). Column 3 shows the amino acid sequence identification number for the polypeptide sequence (SEQ. ID. NO. (P1)). Column 4 shows the NCBI designation, clone identification, or polypeptide fragment identification (Clone ID). Column 5 sets forth the amino acid sequences of some of the polypeptide fragments designated in column 4 (Sequence Fragment).

Table 2 provides information regarding predicted post-translational modifications to the LRRTM1 polypeptide. Column 1 identifies the amino acid position of the consensus post-translational modification site in the LRRTM1 polypeptide; the N-terminal methionine residue is designated position 1. Column 2 identifies the amino acid sequence of the consensus post-translational modification site; capital letters designate residues that are more important in the consensus sequence; small letters designate residues that are less important in the consensus sequence. Column 3 identifies the predicted post-translational modification.

Table 3 lists certain predicted characteristics of the polypeptide encoded by clone ID 16552104, identified by hybridization to probe 238815_at. Clone ID 16652104 was assigned to cluster 185918. A cluster is an internally devised reference to a single locus on a human chromosome which comprises one gene and all its variants. The predicted protein length is 522 amino acids and the different peptide coordinates identify amino acid positions in the predicted protein, with the N-terminal methionine residue designated as position 1. “Treevote” is an algorithm that predicts whether a predicted amino acid sequence is secreted. A Treevote of 0.03 indicates a low probability that the protein is secreted. A Treevote of 1.00 indicates a high probability that the protein is secreted. The mature peptide coordinates refer to the coordinates of the amino acid residues of the mature polypeptide after cleavage of the secretory leader or signal peptide sequence. The alternative mature peptide coordinates result from alternative predictions of the signal peptide cleavage site; their presence may, for example, depend on the host used to express the polypeptides. The transmembrane coordinates designate the transmembrane domains of the molecule. The non-transmembrane coordinates refer to the protein segments not located in the membrane; these can include extracellular, cytoplasmic, and luminal sequences. Finally, clone 16552104 has a leucine-rich repeat (LRR) pfam domain.

Table 4 shows the coordinates of predicted functional domains within the predicted protein encoded by clone ID 16552104. The coordinates identify amino acid positions in the predicted protein, starting at the N-terminal methionine residue as position 1. Each of the LRR domains contributes to the backbone of the curved solenoid fold typical of LRR-motif-containing proteins. The backbone lines a horseshoe-shaped binding pocket which participates in intermolecular interactions. The N-terminal beta finger and C-terminal beta loop form interactions at either edge of the binding pocket.

### BRIEF DESCRIPTION OF THE FIGURES

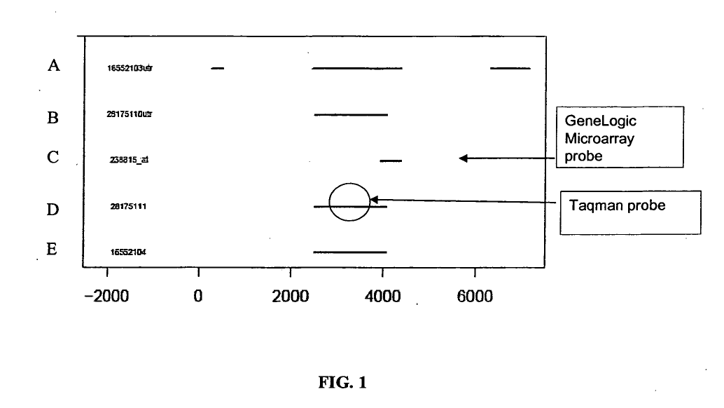

FIG. 1 shows the complete exon map of LRRTM1 including the 5′ and 3′ untranslated regions (UTR) (A and B), the location of microarray hybridization probe 238815_at for LRRTM1 (C), and the complete exon map of the open reading frames of LRRTM1 (D and E). The horizontal axis is a scaled version of the genome which considers all introns to have equal lengths.

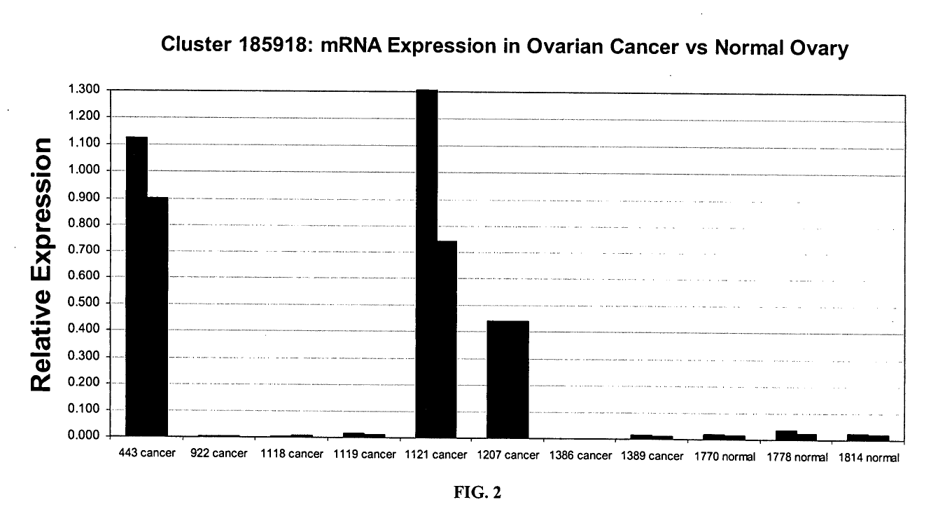

FIG. 2 shows the relative expression of Cluster 185918 mRNA in ovarian cancer and normal adjacent tissue specimens, as determined by real-time PCR.

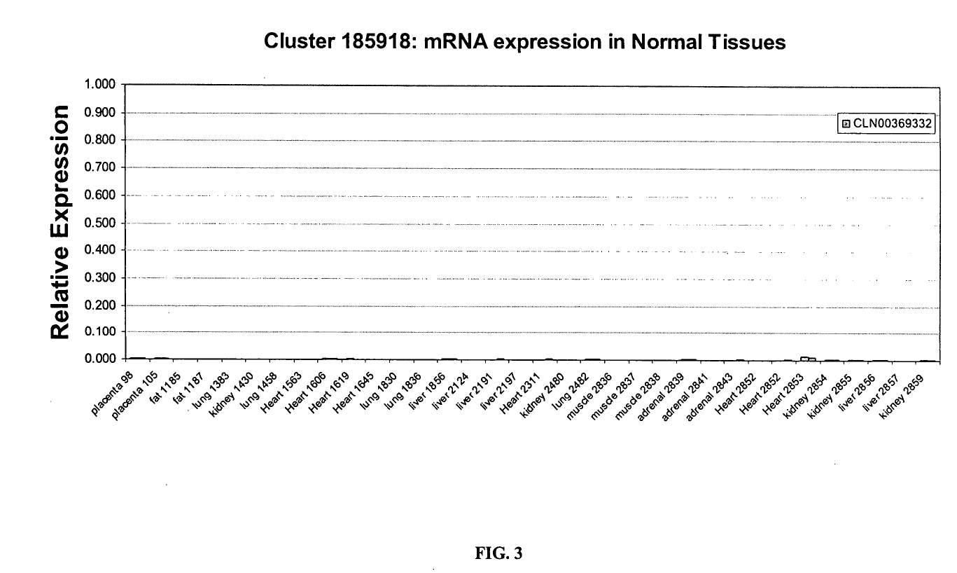

FIG. 3 shows the relative expression of Cluster 185918 mRNA in normal tissue specimens, as determined by real-time PCR.

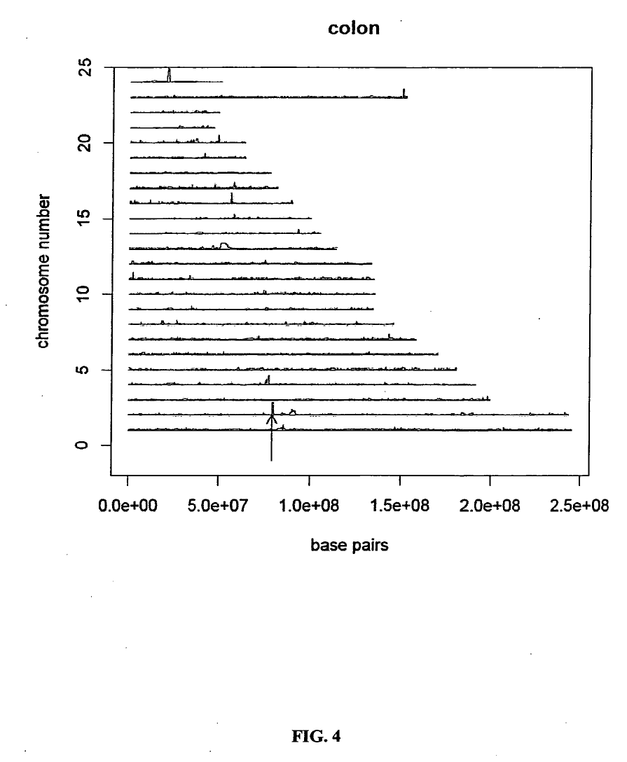

FIG. 4 shows a bioinformatic whole-genome gene expression analysis of a population of colon/colorectal tumor samples in the commercially available GeneLogic database.

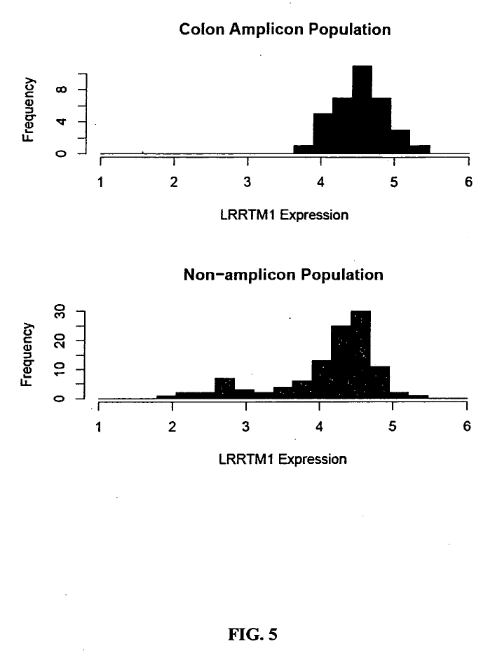

FIG. 5, top panel, shows LRRTM1 expression in the amplicon population of colon/colorectal tumor samples. FIG. 5, bottom panel, shows LRRTM1 expression in the non-amplicon population of colon/colorectal tumor samples.

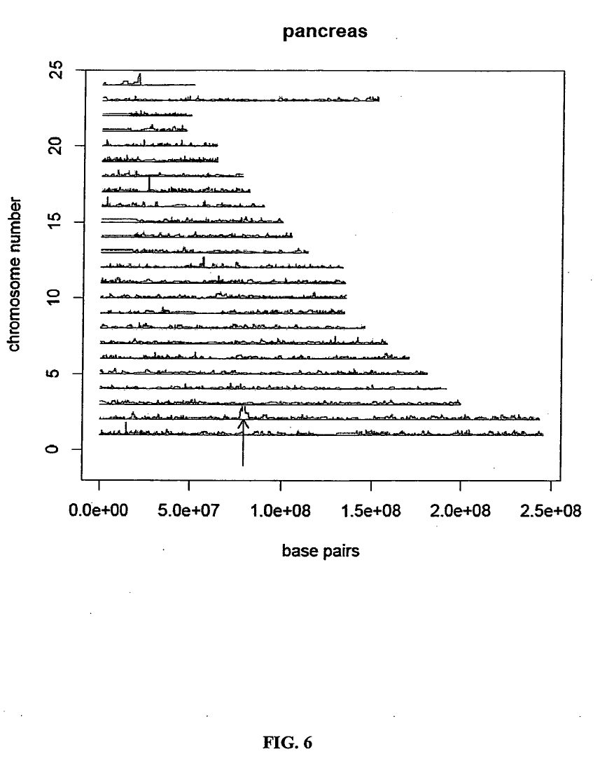

FIG. 6 shows a bioinformatic whole-genome gene expression analysis of a population of pancreatic tumor samples in the commercially available GeneLogic database.

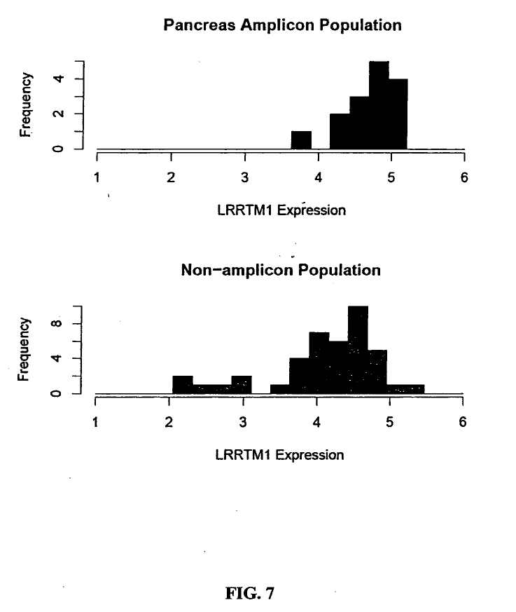

FIG. 7, top panel, shows LRRTM1 expression in the amplicon population of pancreatic tumor samples. FIG. 7, bottom panel, shows LRRTM1 expression in the non-amplicon population of pancreatic tumor samples.

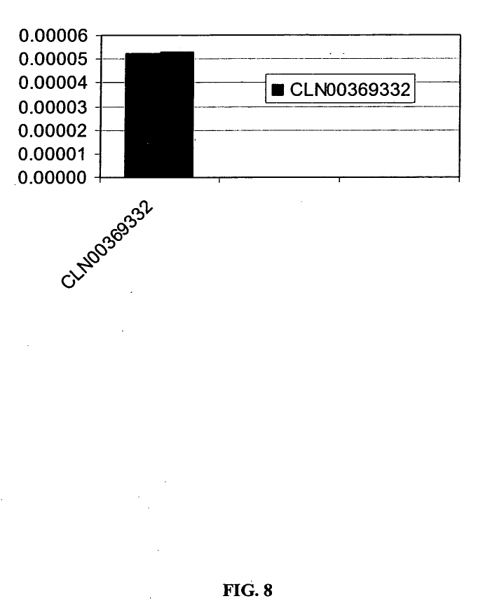

FIG. 8 shows the specificity of real-time PCR primers/probes for LRRTM1. The primers/probes were designed for use in real-time PCR to specifically detect a LRRTM1 open reading frame and were tested with a DNA template encoding LRRTM1.

### DISCLOSURE OF THE INVENTION

The invention provides target polynucleotides and polypeptides useful for diagnosing and treating proliferative disease. It provides compositions comprising LRRTM1. It also provides probes that detect the overexpression of LRRTM1 in cancer. It further provides inhibitors, such as antibodies, that may function as antagonists, and/or may specifically bind to or interfere with the activity of LRRTM1 or fragments of LRRTM1. For example, polypeptides described herein can be used as immunogens to produce specific antibody modulators directed against the polypeptide targets. These antibodies can bind to and inhibit polypeptides on cell surfaces, such as the extracellular or secreted domain of a transmembrane protein, for example, by inducing antibody-dependent cell cytotoxicity (ADCC) or complement-dependent cytotoxicity (CDC), carry a payload, such as a radioisotope or a cytotoxic molecule, or act as antagonist antibodies, for example by affecting ligand/receptor interactions, affecting cofactor interactions, or interfering with cell signaling. The inhibitors of the invention include not only antibodies, but also small molecule drugs, RNAi molecules, ribozymes, antisense molecules, aptamers, soluble receptors, and extracellular fragments of receptors or transmembrane proteins.

LRRTM1 screening assays can identify inhibitors with a desired biologic or therapeutic effect. Modulators of the invention include therapeutic agents that can be used to treat proliferative diseases, such as cancer. The polypeptides and polynucleotides herein are highly expressed in certain tumor tissues compared to normal tissue, especially normal tissues vulnerable to unwanted side effects of drugs. As shown below, microarray hybridization and real-time PCR performed on normal tissue specimens demonstrated that the expression level of LRRTM1 was very low in normal placenta, adipose tissue, lung, kidney, heart, liver, muscle, ovary, colon, and adrenal gland.

### DEFINITIONS

The terms used herein have their ordinary meanings, as set forth below, and can be further understood in the context of the specification.

The terms “polynucleotide,” “nucleic acid,” “nucleic acid molecule,” “nucleic acid sequence,” “polynucleotide sequence,” and “nucleotide sequence” are used interchangeably herein to refer to polymeric forms of nucleotides of any length. The polynucleotides can contain deoxyribonucleotides, ribonucleotides, and/or their analogs or derivatives. Unless specified otherwise, nucleotide sequences shown herein are listed in the 5′ to 3′ direction.

The terms “polypeptide,” “peptide,” and “protein,” used interchangeably herein, refer to a polymeric form of amino acids of any length, which can include naturally-occurring amino acids, coded and non-coded amino acids, chemically or biochemically modified, derivatized, or designer amino acids, amino acid analogs, peptidomimetics, and depsipeptides, and polypeptides having modified, cyclic, bicyclic, depsicyclic, or depsibicyclic peptide backbones. The term includes single chain protein as well as multimers. The term also includes conjugated proteins, fusion proteins, including, but not limited to, glutathione S-transferase (GST) fusion proteins, fusion proteins with a heterologous amino acid sequence, fusion proteins with heterologous and homologous leader sequences, fusion proteins with or without N-terminal methionine residues, pegylated proteins, and immunologically tagged, or his-tagged proteins. The term also includes peptide aptamers.

A “fusion molecule” is a molecule, for example, a polynucleotide or polypeptide, that represents the joining of all or portions of more than one gene or its products. For example, a fusion protein can be the product from splicing strands of recombinant DNA and expressing the hybrid gene. A fusion molecule can be made by genetic engineering, e.g., by removing the stop codon from the DNA sequence of the first protein, then appending the DNA sequence of the second protein in frame. That DNA sequence will then be expressed by a cell as a single protein. Typically this is accomplished by cloning a cDNA into an expression vector in frame with an existing gene.

A “soluble receptor,” or a “soluble form” of a transmembrane protein such as LRRTM1, is a receptor or other polypeptide that lacks a membrane anchor domain, such as a transmembrane domain, present in the full length form of the receptor or transmembrane protein. A soluble receptor or soluble form of a transmembrane protein may be encoded by a naturally-occurring splice variant of a nucleic acid encoding a wild-type transmembrane protein or transmembrane receptor protein in which the transmembrane domain is spliced out of the nucleic acid, and the extracellular domain or any fragment of the extracellular domain of the transmembrane protein or transmembrane receptor protein remain. Alternatively, such soluble forms can be produced by proteolysis of the membrane-spanning form of the receptor or transmembrane protein, thereby releasing all or a portion of the extracellular domain. Soluble receptors can modulate a target protein. They can, for example, compete with wild-type receptors for ligand binding and participate in ligand/receptor interactions, thus modulating the activity of or the number of the receptors and/or the cellular activity downstream from the receptors. This modulation may trigger intracellular responses, for example, signal transduction events which activate cells, signal transduction events which inhibit cells, or events that modulate cellular growth, proliferation, differentiation, and/or death, or induce the production of other factors that, in turn, mediate such activities.

A “biologically active” entity, or an entity having “biological activity,” is one or more entities having structural, regulatory, or biochemical functions of a naturally occurring molecule or any function related to or associated with a metabolic or physiological process. Biologically active polynucleotide fragments are those exhibiting activity similar, but not necessarily identical, to an activity of a polynucleotide of the present invention. The biological activity can include an improved desired activity, or a decreased undesirable activity. For example, an entity demonstrates biological activity when it participates in a molecular interaction with another molecule, such as hybridization, when it has therapeutic value in alleviating a disease condition, when it has prophylactic value in inducing an immune response, when it has diagnostic value in determining the presence of a molecule; or a biologically active fragment of a molecule, or that can be used as a primer in a polymerase chain reaction. A biologically active polypeptide or fragment thereof includes one that can participate in a biological reaction, for example, one that can serve as an epitope or immunogen to stimulate an immune response, such as production of antibodies, or that can participate in stimulating or inhibiting signal transduction by binding to ligands, receptors or other proteins, or nucleic acids; or activating enzymes or substrates.

“Plasma stability” refers to the tendency of a molecule to retain its biological activity in plasma in vivo. It can be determined, for example, by the molecule's bodily absorption, distribution, metabolism, and/or excretion.

The terms “antibody” and “immunoglobulin” refer to a protein, for example, one generated by the immune system, synthetically, or recombinantly, that is capable of recognizing and binding to a specific antigen; antibodies are commonly known in the art. Antibodies may recognize polypeptide or polynucleotide antigens. The term includes active fragments, including for example, an antigen binding fragment of an immunoglobulin, a variable and/or constant region of a heavy chain, a variable and/or constant region of a light chain, a complementarity determining region (CDR), and a framework region. The terms include polyclonal and monoclonal antibody preparations, as well as preparations including hybrid antibodies, altered antibodies, chimeric antibodies, hybrid antibody molecules, F(ab′)₂ and F(ab) fragments; Fv molecules (for example, noncovalent heterodimers), dimeric and trimeric antibody fragment constructs; minibodies, humanized antibody molecules, and any functional fragments obtained from such molecules, wherein such fragments retain specific binding.

A “chimeric” antibody is an antibody or immunoglobulin that contains a variable region that contains an antigen-binding specificity derived from a non-human immunoglobulin and a constant region derived from a human immunoglobulin.

A “humanized” antibody is an antibody or immunoglobulin that contains an antigen-binding specificity derived from a non-human immunoglobulin and the remaining immunoglobulin-derived parts derived from a human immunoglobulin. This term is generally used to refer to an immunoglobulin that has been modified to incorporate sequences from one or more non-human CDRs into one or more human framework regions within the variable domains of a human immunoglobulin. The non-human regions of a humanized antibody may extend beyond the CDRs into the framework regions to achieve the desired antigen-binding properties.

A “fully human” antibody is an antibody produced in a non-human animal that is transgenic at one or more immunoglobulin loci. The one or more transgenic immunoglobulin loci contain sequences from human immunoglobulin loci and deletion of sequences from the non-human animal immunoglobulin loci.

An “epitope” is a molecule to which an antibody binds, which may or may not be a contiguous sequence of amino acid residues in a polypeptide, and which may comprise sugars and/or molecules having other chemical structures.

The term “antibody target” or “cancer target” refers to a polypeptide, polynucleotide, or carbohydrate that can be used as an immunogen in the production of antibodies that specifically bind to such a polypeptide, polynucleotide, or carbohydrate, or a small molecule drug that modulates the activity of such polypeptide, polynucleotide, or carbohydrate.

“Antibody-dependent cell cytotoxicity” (ADCC) is a form of cell mediated cytotoxicity in which an effector cell, such as a lymphocyte, NK cell, granulocyte, neutrophil, eosinophil, basophil, mast cell, or macrophage, mediates the killing of a cell to which an antibody is attached. ADCC can involve humoral and/or cell-dependent mechanisms.

“Complement-dependent cytotoxicity” (CDC) is an adverse effect on a cell that can result from activation of the complement pathway. It includes actions mediated through the classical complement pathway.

The term “binds specifically,” in the context of antibody binding, refers to high avidity and/or high affinity binding of an antibody to a specific epitope. Hence, an antibody that binds specifically to one epitope (a “first epitope”) and not to another (a “second epitope”) is a “specific antibody.” An antibody specific to a first epitope may cross react with and bind to a second epitope if the two epitopes share homology or other similarity.

The term “binds specifically,” in the context of a polynucleotide, refers to hybridization under stringent conditions. Conditions that increase stringency of both DNA/DNA and DNA/RNA hybridization reactions are widely known and published in the art. See, for example, Sambrook, J., et al. (2000) Molecular Cloning, A Laboratory Manual. 3rd ed. Cold Spring Harbor Laboratory Press.

An “isolated,” “purified,” “substantially isolated,” or “substantially purified” molecule (such as a polypeptide, polynucleotide, or antibody) is one that has been manipulated to exist in a higher concentration than in nature. For example, a subject antibody is isolated, purified, substantially isolated, or substantially purified when at least about 10%, or 20%, or 40%, or 50%, or 70%, or 90% of non-subject-antibody materials with which it is associated in nature have been removed. As used herein, an “isolated,” “purified,” “substantially isolated,” or “substantially purified” molecule includes recombinant molecules.

A “host cell” is an individual cell or cell culture which can be or has been a recipient of any recombinant vector(s) or isolated polynucleotide. Host cells include prokaryotic cells and eukaryotic cells. Host cells also include progeny of a single host cell, and the progeny may not necessarily be completely identical (in morphology or in total DNA complement) to the original parent cell due to natural, accidental; or deliberate mutation and/or change. A host cell includes cells transfected or infected in vivo or in vitro with a recombinant vector or a polynucleotide of the invention. A host cell which comprises a recombinant vector of the invention may be called a “recombinant host cell.”

“Patient,” “individual,” “host,” and “subject” are used interchangeably herein to refer to mammals, including, but not limited to, rodents, simians, humans, felines, canines, equines, bovines, porcines, ovines, caprines, mammalian laboratory animals, mammalian farm animals, mammalian sport animals, and mammalian pets.

A “body fluid sample,” or “patient sample” is any biological specimen derived from a patient; the term includes, but is not limited to, biological fluids such as blood, serum, plasma, urine, cerebrospinal fluid, tears, saliva, lymph, dialysis fluid, lavage fluid, semen, and other liquid samples, as well as cell and tissues of biological origin. The term also includes cells or cells derived therefrom and the progeny thereof, including cells in culture, cell supernatants, and cell lysates. It further includes organ or tissue culture-derived fluids, tissue biopsy samples, tumor biopsy samples, stool samples, and fluids extracted from physiological tissues, as well as cells dissociated from solid tissues, tissue sections, and cell lysates. This definition encompasses samples that have been manipulated in any way after their procurement, such as by treatment with reagents, solubilization, or enrichment for certain components, such as polynucleotides or polypeptides. Also included in the term are derivatives and fractions of body fluid samples. A body fluid sample may be used in a diagnostic, prognostic, or other monitoring assay.

The term “receptor” refers to a polypeptide that binds to a specific ligand. The ligand is usually an extracellular molecule which, upon binding to the receptor, usually initiates a cellular response such as initiation of a signal transduction pathway.

The term “ligand” refers to a molecule that binds to a specific site on another molecule, usually a receptor.

The term “modulate” refers to the production, either directly or indirectly, of an increase or a decrease, a stimulation, inhibition, interference, or blockage in a measured activity when compared to a suitable control. A “modulator” of a polypeptide or polynucleotide or an “agent” are terms used interchangeably herein to refer to a substance that affects, for example, increases, decreases, stimulates, inhibits, interferes with, or blocks a measured activity of the polypeptide or polynucleotide, when compared to a suitable control.

The term “agonist” refers to a substance that mimics or enhances the function of an active molecule. Agonists include, but are not limited to, antibodies, growth factors, cytokines, lymphokines, small molecule drugs, hormones, and neurotransmitters, as well as analogues and fragments thereof.

The term “antagonist” refers to a molecule that interferes with the activity or binding of another molecule such as an agonist, for example, by competing for the one or more binding sites of an agonist, but does not induce an active response.

“Diagnosis,” as used herein, is the determination of the presence or absence of a disease or the propensity for contracting a disease. Assays for LRRTM1, as described herein, can be used to diagnose certain cancers.

“Prognosis,” as used herein, is the determination of the probable course or outcome of a disease. It may include a determination of the likelihood of recovery, a prediction of disease symptoms, and/or a prediction of the likely nature and course of the disease.

“Treatment,” as used herein, covers any administration or application of remedies for disease in a mammal, including a human, and includes inhibiting the disease, arresting its development, or relieving the disease, for example, by causing regression, or restoring or repairing a lost, missing, or defective function; or stimulating an inefficient process.

“Prophylaxis,” as used herein, includes preventing a disease from occurring or recurring in a subject that may be predisposed to the disease but is not currently symptomatic. Treatment and prophylaxis can be administered to an organism, or to a cell in vivo, in vitro, or ex vivo, and the cell subsequently administered to the subject.

A “pharmaceutically acceptable carrier” refers to a non-toxic solid, semisolid or liquid filler, diluent, encapsulating material, formulation auxiliary, or excipient of any conventional type. A pharmaceutically acceptable carrier is non-toxic to recipients at the dosages and concentrations employed and is compatible with other ingredients of the formulation.

A “composition” herein refers to a mixture that usually contains a carrier, such as a pharmaceutically acceptable carrier or excipient that is conventional in the art and which is suitable for administration into a subject for therapeutic, diagnostic, or prophylactic purposes. It may include a cell culture in which the polypeptide or polynucleotide is present in the cells or in the culture medium. For example, compositions for oral administration can form solutions, suspensions, tablets, pills, capsules, sustained release formulations, oral rinses, or powders.

A “vaccine” is a preparation that produces or artificially increases immunity to a particular disease. It may, for example, be comprised of killed microorganisms, living attenuated organisms, or living virulent organisms that is administered to produce or artificially increase immunity to a particular disease. It includes a preparation containing weakened or dead microbes of the kind that cause a particular disease, administered to stimulate the immune system to produce antibodies against that disease. The term includes nucleic acid and polypeptide vaccines.

“Disease” refers to any condition, infection, disorder, or syndrome that requires medical intervention or for which medical intervention is desirable. Such medical intervention can include treatment, diagnosis, and/or prevention.

“Cancer” is any abnormal cell or tissue growth, for example, a tumor, whether malignant, pre-malignant, or non-malignant. It is characterized by uncontrolled proliferation of cells that may or may not invade the surrounding tissue and, hence, may or may not metastasize to new body sites. Cancer encompasses carcinomas, which are cancers of epithelial cells; carcinomas include squamous cell carcinomas, adenocarcinomas, melanomas, and hepatomas. Cancer also encompasses sarcomas, which are tumors of mesenchymal origin; sarcomas include osteogenic sarcomas, leukemias, and lymphomas. Cancers may involve one or more neoplastic cell type.

### Target Molecule LRRTM1

The LRRTM1 nucleic acid sequence, designated NM_{—}178839.3 in the NCBI database, encodes a 522-amino acid protein, designated NP_{—}849161.1 in the NCBI database. The structural information for LRRTM1 in the Structural Classification of Proteins database (SCOP; available at http://scop.mrc-lmb.cam.ac.uk/scop) reveals that LRRTM1 is a member of the class of alpha and beta proteins (a/b), which comprises parallel beta sheets. LRRTM1 is also a member of the fold known as leucine-rich repeat, LRR, which comprises right-handed beta-alpha superhelix structures. LRRTM1 belongs to three different superfamilies, the RNI-like superfamily, which comprises a regular structure consisting of similar repeats; the L domain-like superfamily, which comprises a less regular structure consisting of variable repeats; and the outer arm dyne in light chain 1 superfamily, which comprises one or more beta-beta-alpha superhelix structures. The LRRTM1 transcript and polypeptide produced from the transcript are present in humans in vivo and are physiologically relevant, as described herein in further detail.

Accordingly, the invention provides an isolated first nucleic acid molecule comprising a first polynucleotide sequence encoding a polypeptide, a complement thereof, an isolated polypeptide encoded by a polynucleotide, wherein the polypeptide comprises an amino acid sequence chosen from the Tables, Figures, and Sequence Listing, or a biologically active fragment thereof, wherein the polypeptide is other than a full-length LRRTM1.

The invention provides polynucleotide sequences of the open reading frames that encode polypeptides of the invention (SEQ. ID. NO. (N1)). SEQ. ID. NO.:27 represents the complete nucleotide sequence of LRRTM1, identified as NP_{—}849161.1:NM_{—}178839.3 in the NCBI database; SEQ. ID. NO.:13 represents the nucleotide sequence of the coding region of clone 16552104:16552103. The invention further provides nucleotide sequences encoding polypeptide fragments of LRRTM1, represented by SEQ. ID. NOS.:1-12, used as described herein.

The invention also provides amino acid sequences of polypeptides of the invention (SEQ ID NO. (P1)). SEQ. ID. NO.:28 represents the amino acid sequence of LRRTM1, identified as NP_{—}849161.1:NM_{—}178839.3 in the NCBI database. According to Swiss-Prot annotation Q86UE2, Entry Version 29 modified on Sep. 5, 2006, residues 1-34 are believed to represent the signal sequence, residues 35-522 are believed to represent mature full length LRRTM1, residues 35 to 427 are believed to represent the extracellular domain, residues 428-448 are believed to represent the transmembrane domain, and residues 449-522 are believed to represent the cytoplasmic domain. SEQ. ID. NOS.:14-25 represent the amino acid sequences of fragments of LRRTM1, used as described herein.

Bioinformatic Database Analysis and Real Time PCR

LRRTM1 nucleic acid molecules and their encoded proteins, shown in the Tables, Figures, and Sequence Listing, may serve as antibody targets of the invention. They may also serve as target molecules for the production of modulators such as antibodies. As shown, for example, in Examples 1 and 2, and FIGS. 2 and 3, selected tumor tissues expressed higher levels of LRRTM1 mRNA compared to normal tissue. FIG. 2 shows that LRRTM1 is overexpressed in certain ovarian tumors, for example, adenocarcinomas, compared to normal ovarian tissues, while FIG. 3 shows that LRRTM1 is expressed at low to undetectable levels in a wide range of normal tissues.

Quantitative real time-PCR, as set forth in Example 2, was used to quantitate mRNA expression. Specific primers and probes were designed for the detection of exon 2 of LRRTM1. As shown in FIG. 2, LRRTM1 is overexpressed in certain ovarian cancers. In FIG. 2, sample “443 cancer” is an adenocarcinoma, metastasis to the omentum; “992 cancer” is an adenocarcinoma; “1118 cancer,” “1119 cancer,” and “1207 cancer,” are each a papillary serous adenocarcinoma; and “1121 cancer” is a papillary serous adenocarcinoma, grade 2. In addition, FIG. 3 shows that LRRTM1 is expressed at a low or undetectable level in several normal tissues including heart, lung, kidney, placenta, liver, adipose tissue, muscle, and adrenal gland. None of the normal ovarian tissue tested demonstrated the same level of overexpression seen in the cancerous tissue.

FIGS. 4 and 5 show that LRRTM1 is also overexpressed in a subset of colon/colorectal tumors. FIG. 4 shows a bioinformatic whole-genome gene expression analysis of a population of colon/colorectal tumor samples in the commercially available database GeneLogic. Each horizontal line corresponds to a human chromosome; the chromosome number is indicated on the vertical axis. The height of the vertical tick marks on each chromosome indicates the relative amount of expression in a cluster of adjacent genes in a subpopulation of colon/colorectal cancer samples. The arrow marks the location of LRRTM1 on chromosome 2, and the peak (tall tick mark) indicates that there is overexpression of a cluster of genes near this locus in a subpopulation of colon/colorectal cancer samples. That subpopulation is referred to as the “amplicon population,” as indicated in FIG. 5 (below). Gene clusters near the LRRTM1 locus that do not show overexpression, as evidenced by short tick marks, are referred to as the “non-amplicon population” as indicated in FIG. 5 (below).

FIG. 5, top panel, shows LRRTM1 expression in the amplicon population of colon/colorectal tumor samples. FIG. 5, bottom panel, shows LRRTM1 expression in the non-amplicon population of colon/colorectal tumor samples. On the vertical axis, “frequency” indicates the number of colon cancer samples; on the horizontal axis, “LRRTM1 expression” indicates the natural log of the gene expression intensity. Comparison of the expression profile of FIG. 5, top panel, to that of FIG. 5, bottom panel, shows that expression of LRRTM1 in the amplicon population is shifted to the right, and is concentrated in the high range of expression values of the non-amplicon population.

FIGS. 6 and 7 show that LRRTM1 is also overexpressed in a subset of pancreatic tumors. FIG. 6 shows a bioinformatic whole-genome gene expression analysis of a population of pancreatic tumor samples in the commercially available GeneLogic database. Each horizontal line corresponds to a human chromosome; the chromosome number is indicated on the vertical axis. The height of the vertical tick marks on each chromosome indicates the relative amount of expression in a cluster of adjacent genes in a subpopulation of pancreatic cancer samples. The arrow marks the location of LRRTM1 on chromosome 2, and the peak (tall tick mark) indicates that there is overexpression of a cluster of genes near this locus in a subpopulation of pancreatic cancer samples. That subpopulation is referred to as the “amplicon population” as indicated in FIG. 7 (below). Gene clusters near the LRRTM1 locus that do not show overexpression, as evidenced by short tick marks, are referred to as the “non-amplicon population” as indicated in FIG. 7 (below).

FIG. 7, top panel, shows LRRTM1 expression in the amplicon population of pancreatic tumor samples. FIG. 7, bottom panel, shows LRRTM1 expression in the non-amplicon population of pancreatic tumor samples. On the vertical axis, “frequency” indicates the number of pancreatic cancer samples; on the horizontal axis, “LRRTM1 expression” indicates the natural log of the gene expression intensity. Comparison of the expression profile of FIG. 7, top panel, to that of FIG. 7, bottom panel, shows that expression of LRRTM1 in the amplicon population is shifted to the right, and is concentrated in the high range of expression values of the non-amplicon population.

LRRTM1 Antibody Target

LRRTM1 is an antibody target which corresponds to a probe that exhibited a “hit” when hybridized to the cRNA on a FivePrime microarray chip. The specific individual probe hit was identified as 238815_at, and the fragment ID, also referred to as the chip ID, corresponding to the tissue RNA, was identified as belonging to gene cluster 185918, in which it was observed to be located. This cluster represents a group of human cDNA clones which map to a single locus on the human chromosome.

LRRTM1 is a prophylactic or therapeutic target for cancer, since it is predicted to be a transmembrane protein and it is overexpressed on the surface of certain cancer tissues compared to normal tissues. Transmembrane proteins extend into or through the cell membrane's lipid bilayer; they can span the membrane once, or more than once. Transmembrane proteins, having part of their molecules on either side of the bilayer, have many and widely variant biological functions. Transmembrane proteins are often involved in cell signaling events; they can comprise signaling molecules, or can interact with signaling molecules. Extracellular domains of transmembrane proteins may be cleaved to produce soluble receptors.

Antibodies are particularly suited to be used as therapeutic agents when their targets are transmembrane proteins expressed on the surface of cancer cells. Thus, in one aspect of the invention, the nucleic acids and proteins are antibody targets or markers or biomarkers identified by binding to an antibody. Among the antibody targets of the invention are polypeptides encoded by the gene LRRTM1 (Lauren, J. et al., (2003) Genomics 81:411-421; NP_{—}849161.1:NM_{—}178839.3 in the NCBI database). This gene encodes a protein comprising ten leucine-rich repeat domains. The protein has been implicated in embryonic brain development and maintenance, but has not been implicated in ovarian, pancreatic, or colon/colorectal tumor formation, growth, metastasis, or other aspect of tumor biology.

Antibodies binding to the extracellular domain of LRRTM1 are therapeutic for cancers, including ovarian cancer, pancreatic cancer, and colon/colorectal cancer. Such antibodies can be used as monotherapy if they mediate ADCC or CDC, or if they modify the underlying function of the target molecule (in this case, LRRTM1 function). Anti-LRRTM1 antibodies can also be used in the form of antibody conjugates to directly deliver cancer agents with a lethal effect on the tumor. Such agents include radionuclides, toxins, and chemotherapeutics.

Antibodies binding to the extracellular domain of LRRTM1 can also advantageously be used for the detection of soluble forms of LRRTM1 in fluids comprising biological materials, including, but not limited to, body fluids obtained from subjects for diagnostic and/or prognostic purposes. Such fluids include, but are not limited to, blood, serum, plasma, cerebrospinal fluid, urine, saliva, sputum, pleural effusions, lavage fluids (such as bronchial vaginal, or cervical washes), etc.

Anti-LRRTM1 antibodies can also be used in combination with standard chemotherapeutic or radiation regimens to treat cancers. In this case, anti-LRRTM1 antibodies can act to sensitize the cancer cells to chemotherapy or radiation, allowing for more efficient tumor killing. Alternatively, anti-LRRTM1 antibodies can act in synergy with chemotherapy or radiation treatment, such that lower doses of either may be used, decreasing the overall toxicity to normal cells while maintaining equivalent efficacy in treating the tumor.

Antibodies having a therapeutic effect on cancers include those binding to LRRTM1 amino acid sequences involved in LRRTM1 function. Such amino acid sequences include, for example, those corresponding to one or more of the leucine-rich repeat domains, the cysteine-rich N-terminal domain and/or the C-terminal flanking region of the extracellular domain. Such amino acid sequences may also include those that are post-translationally modified, for example, by glycosylation, for example, N-glycosylation.

Such antibodies include those binding to LRRTM1 amino acids 62-85, HNLSGLLGLSLRYNSLSELRAGQF (SEQ. ID. NO.:14), which comprise leucine-rich repeat 1. The invention provides antibodies that bind to this sequence and/or to any epitope of six or more consecutive amino acids contained within the sequence.

Such antibodies include those binding to LRRTM1 amino acids 86-109, TGLMQLTWLYLDHNHICSVQGDAF (SEQ. ID. NO.:15), which comprise leucine-rich repeat 2. The invention provides antibodies that bind to this sequence and/or to any epitope of six or more consecutive amino acids contained within the sequence. Such antibodies include those binding to LRRTM1 amino acids 110-133, QKLRRVKELTLSSNQITQLPNTTF (SEQ. ID. NO.:16), which comprise leucine-rich repeat 3. The invention provides antibodies that bind to this sequence and/or to any epitope of six or more consecutive amino acids contained within the sequence.

Such antibodies include those binding to LRRTM1 amino acids 134-157, RPMPNLRSVDLSYNKLQALAPDLF (SEQ. ID. NO.:17), which comprise leucine-rich repeat 4. The invention provides antibodies that bind to this sequence and/or to any epitope of six or more consecutive amino acids contained within the sequence.

Such antibodies include those binding to LRRTM1 amino acids 158-181, HGLRKLTTLHMRANAIQFVPVRIF (SEQ. ID. NO.:18), which comprise leucine-rich repeat 5. The invention provides antibodies that bind to this sequence and/or to any epitope of six or more consecutive amino acids contained within the sequence.

Such antibodies include those binding to LRRTM1 amino acids 182-205, QDCRSLKFLDIGYNQLKSLARNSF (SEQ. ID. NO.:19), which comprise leucine-rich repeat 6. The invention provides antibodies that bind to this sequence and/or to any epitope of six or more consecutive amino acids contained within the sequence.

Such antibodies include those binding to LRRTM1 amino acids 206-229, AGLFKLTELHLEHNDLVKVNFAHF (SEQ. ID. NO.:20), which comprise leucine-rich repeat 7. The invention provides antibodies that bind to this sequence and/or to any epitope of six or more consecutive amino acids contained within the sequence.

Such antibodies include those binding to LRRTM1 amino acids 230-253, PRLISLHSLCLRRNKVAIVVSSLD (SEQ. ID. NO.:21), which comprise leucine-rich repeat 8. The invention provides antibodies that bind to this sequence and/or to any epitope of six or more consecutive amino acids contained within the sequence.

Such antibodies include those binding to LRRTM1 amino acids 253-276, DWVWNLEKMDLSGNEIEYMEPHVF (SEQ. ID. NO.:22), which comprise leucine-rich repeat 9. The invention provides antibodies that bind to this sequence and/or to any epitope of six or more consecutive amino acids contained within the sequence.

Such antibodies include those binding to LRRTM1 amino acids 277-300, ETVPHLQSLQLDSNRLTYIEPRIL (SEQ. ID. NO.:23), which comprise leucine-rich repeat 10. The invention provides antibodies that bind to this sequence and/or to any epitope of six or more consecutive amino acids contained within the sequence.

Such antibodies include those binding to LRRTM1 amino acids 34-58, AAPSGCPQLCRCEGRLLYCEALNLT (SEQ. ID. NO.:24), which comprise a fragment of the extracellular domain, N-terminal to the leucine-rich repeat domains. The invention provides antibodies that bind to this sequence and/or to any epitope of six or more consecutive amino acids contained within the sequence.

Such antibodies include those binding to LRRTM1 amino acids 306-346, LTSITLAGNLWDCGRNVCALASWLNNFQGRYDGNLQCASPE (SEQ. ID. NO.:25), which comprise a fragment of the extracellular domain, C-terminal to the leucine-rich repeat domains. The invention provides antibodies that bind to this sequence and/or to any epitope of six or more consecutive amino acids contained within the sequence.

LRRTM1 Protein Domains

The LRRTM1 sequences of the invention encompass a variety of different nucleic acids and polypeptides with different structures and functions, embodied in different molecular domains. They can encode or comprise polypeptides belonging to different protein families, for example, those described by the Pfam database. The Pfam system is an organization of protein sequence classification and analysis, based on conserved protein domains; it can be publicly accessed in a number of ways, for example, at http://Pfam.wust1.edu. Protein domains are portions of proteins that have a tertiary structure and sometimes have enzymatic or binding activities; multiple domains can be connected by flexible polypeptide regions within a protein. Pfam domains can comprise the N-terminus or the C-terminus of a protein, or can be situated at any point in between. The Pfam system identifies protein families based on these domains and provides an annotated, searchable database that classifies proteins into families (Bateman, A., et al. (2000) Nucleic Acids Research 30:276-280).

Sequences of the invention can encode or be comprised of one or more than one Pfam. Sequences encompassed by the invention include, but are not limited to, the polypeptide and polynucleotide sequences of the molecules shown in the Tables, Figures and Sequence Listing and corresponding molecular sequences found at all developmental stages of an organism. Sequences of the invention can comprise genes or gene segments designated in the Tables, Figures, and Sequence Listing, and their RNA and polypeptide gene products. They also include variants of those presented in the Tables, Figures, and Sequence Listing that are present in the normal physiological state, for example, variant alleles such as SNPs, splice variants, as well as variants that are affected in pathological states, such as disease-related mutations or sequences with alterations that lead to pathology, and variants with conservative amino acid changes. Some sequences of the invention are categorized below with respect to one or more protein family. Any given sequence can belong to one or more than one category.

Polypeptide Expression

The LRRTM1 polypeptides described herein can be expressed using methods known in the art. Cell-based methods and cell-free methods are suitable for producing polypeptides of the invention. The use of the polymerase chain reaction has been described Saiki et al., Nature, 324: 163-166 (1986) and current techniques have been reviewed (Sambrook et al., 2000; McPherson et al. (2000) PCR Basics: From Background to Bench; Springer, Verlag; Dieffenbach and Dveksler, (1995) PCR Primer: A Laboratory Manual; Cold Spring Harbor Laboratory Press). Cell-based methods generally involve introducing a nucleic acid construct into a host cell in vitro and culturing the host cell under conditions suitable for expression, then harvesting the polypeptide, either from the culture medium or from the host cell, (for example, by disrupting the host cell), or both, as described in detail above. The invention also provides methods of producing a polypeptide using cell-free in vitro transcription/translation methods, which are well known in the art.

The LRRTM1 polypeptides can be recovered and purified from recombinant cell cultures by well-known methods, including ammonium sulfate or ethanol precipitation, acid extraction, anion or cation exchange chromatography, phosphocellulose chromatography, hydrophobic interaction chromatography, affinity chromatography, hydroxylapatite chromatography, and lectin chromatography, for example, as described by Deutscher, M. P., et al., eds. (1990) Guide to Protein Purification: Methods in Enzymology. (Methods in Enzymology Series, Vol. 182). Acad. Press. By way of example, high performance liquid chromatography (HPLC) can be employed for purification. LRRTM1 polypeptides include products purified from natural sources, including bodily fluids, tissues and cells, whether directly isolated or cultured; products of chemical synthetic procedures; and products produced by recombinant techniques from a prokaryotic or eukaryotic host, including, for example, bacterial, yeast, higher plant, insect, and mammalian cells. Depending upon the host employed in a recombinant production procedure, the polypeptides of the present invention may be glycosylated or may be non-glycosylated. In addition, polypeptides of the invention may also include an initial modified methionine residue, in some cases as a result of host-mediated processes. Thus, it is well known in the art that the N-terminal methionine encoded by the translation initiation codon generally is removed with high efficiency from any protein after translation in eukaryotic cells. While the N-terminal methionine on most proteins also is efficiently removed in most prokaryotes, for some proteins this prokaryotic removal process is inefficient, depending on the nature of the amino acid to which the N-terminal methionine is covalently linked.

Typically, a heterologous polypeptide, whether modified or unmodified, may be expressed on its own, as described above, or as a fusion protein, and may include not only secretion signals, but also a secretory leader sequence. A leader sequence comprises a sequence of amino acid residues, beginning at amino acid residue 1 located at the amino terminus of the polypeptide, and extending to a cleavage site, which, upon proteolytic cleavage, results in formation of a mature protein. Leader sequences are generally hydrophobic and have some positively charged residues. Leader sequences can be natural or synthetic, heterologous, or homologous with the protein to which they are attached. A secretory leader is a leader sequence that directs a protein to be secreted from the cell. A secretion signal sequence can be naturally occurring or it can be engineered.

A secretory leader sequence of the invention may direct certain proteins to the ER. The ER separates the membrane-bound proteins from other proteins. Once localized to the ER, proteins can be further directed to the Golgi apparatus for distribution to vesicles; including secretory vesicles; the plasma membrane, lysosomes, and other organelles.

Proteins targeted to the ER by a secretory leader sequence can be released into the extracellular space as a secreted protein. Secreted proteins are generally capable of being directed to the endoplasmic reticulum (ER), secretory vesicles, or the extracellular space as a result of a secretory leader, signal peptide, or leader sequence. They may be released into the extracellular space, for example, by exocytosis or proteolytic cleavage, regardless of whether they comprise a signal sequence. A secreted protein may in some circumstances undergo processing to a mature polypeptide. Secreted proteins may comprise leader sequences of amino acid residues, located at the amino terminus of the polypeptide and extending to a cleavage site, which, upon proteolytic cleavage, result in the formation of a mature protein.

In addition, vesicles containing secreted proteins can fuse with the cell membrane and release their contents into the extracellular space in a process called exocytosis. Exocytosis can occur constitutively or in response to a triggering signal. In the latter case, the proteins may be stored in secretory vesicles (or secretory granules) until exocytosis is triggered. Similarly, proteins residing on the cell membrane can also be secreted into the extracellular space by proteolytic cleavage of a linker holding the protein to the membrane.

Additionally, peptide moieties and/or purification tags may be added to the polypeptide to facilitate purification. Such regions may be removed prior to final preparation of the polypeptide. The addition of peptide moieties to polypeptides to engender secretion or excretion, to improve stability, and to facilitate purification, among other reasons, are familiar and routine techniques in the art. Suitable purification tags include, for example, V5, polyhistidines, avidin, and biotin.

Protein expression systems known in the art can produce fusion proteins that incorporate the polypeptides of the invention. LRRTM1 fusion proteins can facilitate production, secretion, and/or purification. They can confer a longer half-life when administered to an animal. Suitable chemical moieties for derivatization of a heterologous polypeptide include, for example, polymers, such as water soluble polymers, the constant domain of immunoglobulins, all or part of human serum albumin; fetuin A; fetuin B; a leucine zipper domain; a tetranectin trimerization domain; mannose binding protein (also known as mannose binding lectin), for example, mannose binding protein 1; and an Fc region, as described herein and further described in U.S. Pat. No. 6,686,179, and U.S. Application Nos. 60/589,788 and 60/654,229. Conjugated proteins, such as polyethylene glycol conjugates, are also provided. Such modified polypeptides can show, for example, enhanced activity or increased stability. In addition, they may be purified in higher yields and show better solubility than the corresponding natural polypeptide, at least under certain purification and storage conditions.

### Kits

Detection of cancer cell-specific biomarkers provides an effective cancer screening strategy. Early detection provides not only early diagnosis, but also the ability to screen for polymorphism and detect post-operative residual tumor cells and occult metastases, an early indicator of tumor recurrence. Early detection of cancer cell-specific biomarkers can thus improve survival in patients before diagnosis, while undergoing treatment, and while in remission.

LRRTM1 is overexpressed in certain cancer tissues from certain cancer patients. Since LRRTM1 is not normally expressed at high levels in certain tissues of healthy, mature, non-pregnant adults, the presence of LRRTM1 can be used as a diagnostic or prognostic marker for cancer, such as in identifying a patient population appropriate for treatment. Diagnostic antibodies can be used in a number of ways, including but not limited to ELISA, Western blot, immunofluorescence, or immunohistochemistry, for these purposes.

Microarrays comprising probes that detect overexpression of LRRTM1 in cancer tissues can provide a tool for cancer diagnosis. The microarrays and probes can be used to diagnose diseases characterized by aberrant expression of LRRTM1. Thus, probes that detect overexpression of LRRTM1 in ovarian, pancreatic, and/or colon/colorectal cancers can improve the diagnosis of those cancers.

The invention provides methods for diagnosing disease states based on the detected presence and/or level of LRRTM1 polynucleotides, polypeptides, or antibodies in a biological sample, and/or the detected presence and/or level of a biological activity of the polynucleotide or polypeptide. These detection methods can be provided as part of a kit. Thus, the invention further provides kits for detecting the presence and/or a level of a polynucleotide, polypeptide, or antibody of interest in a biological sample.

Where the kit provides for polynucleotide detection, it can include one or more nucleotides that hybridize specifically to an LRRTM1 nucleotide of interest. Where the kit provides for polypeptide detection, it can include one or more specific antibodies. In some embodiments, the antibody specific to the polypeptide of interest is detectably labeled. In other embodiments, the antibody specific to the polypeptide is not labeled; instead, a second, detectably labeled antibody is provided that binds to the specific antibody. The kit may further include blocking reagents, buffers, and reagents for developing and/or detecting the detectable marker. The kit may further include instructions for use, controls, and interpretive information.

The invention also provides for therapeutic kits with unit doses of an active agent. In some embodiments, the agent is provided in oral or injectable doses, as described in further detail below. Such kits can comprise containers containing the unit doses and an informational package insert describing the use and attendant benefits of the drugs in treating a condition of interest. In an embodiment, the invention provides a kit pharmaceutical pack comprising one or more containers filled with one or more effective doses of the pharmaceutical LRRTM1 compositions of the invention. The containers may be associated with a notice in a form prescribed by a governmental agency regulating the manufacture, use, or sale for human administration.

### Assay Measurement Strategies

Numerous methods and devices are well known to the skilled artisan for the detection and analysis of polypeptides for diagnostic and prognostic purposes. With regard to polypeptides or proteins in patient test samples, immunoassay devices and methods are often used. See, e.g., U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792, each of which is hereby incorporated by reference in its entirety, including all tables, Figures and claims. These devices and Figures methods can utilize labeled molecules in various sandwich, competitive, or non-competitive assay formats, to generate a signal that is related to the presence or amount of an analyte of interest. Additionally, certain methods and devices, such as biosensors and optical immunoassays, may be employed to determine the presence or amount of analytes without the need for a labeled molecule. See, e.g., U.S. Pat. Nos. 5,631,171; and 5,955,377, each of which is hereby incorporated by reference in its entirety, including all tables, Figures and claims. One skilled in the art also recognizes that robotic Figures instrumentation including, but not limited to, Beckman Access, Abbott AxSym, Roche ElecSys, Dade Behring Stratus systems are among the immunoassay analyzers that are capable of performing the immunoassays taught herein.

In certain embodiments, detection methods are immunoassays. Certain other detection methods include, for example, those that are well known to those skilled in the art (such as the measurement of marker RNA levels). The presence or amount of a polypeptide is generally determined using one or more antibodies that bind to the polypeptide of interest, and followed by detecting binding of polypeptides to the antibody(ies). Any suitable immunoassay may be utilized, for example, enzyme-linked immunoassays (ELISA), radioimmunoassays (RIAs), competitive binding assays, and the like. Specific immunological binding of the antibody to the marker can be detected directly or indirectly. Direct labels include fluorescent or luminescent tags, metals, dyes, radionuclides, and the like, attached to the antibody. Indirect labels include various enzymes well known in the art, such as alkaline phosphatase, horseradish peroxidase and the like.

The use of immobilized antibodies in order to bind a polypeptide of interest for detection is also contemplated by the present invention. Antibodies may be immobilized onto a variety of solid supports, such as magnetic or chromatographic matrix particles, the surface of an assay place (such as microtiter wells), pieces of a solid substrate material or membrane (such as plastic, nylon, paper), and the like. An assay strip could be prepared by coating the antibody or a plurality of antibodies in an array on solid support. This strip could then be dipped into the test sample and then processed quickly through washes and detection steps to generate a measurable signal, such as a colored spot.

The analysis of a plurality of polypeptides may be carried out separately or simultaneously with one test sample. For separate or sequential assay of markers, suitable apparatuses include clinical laboratory analyzers such as the ElecSys (Roche), the AxSym (Abbott), the Access (Beckman), the ADVIA® CENTAUR® (Bayer) immunoassay systems, the NICHOLS ADVANTAGE® (Nichols Institute) immunoassay system, etc. In certain embodiments, apparatuses or protein chips perform simultaneous assays of a plurality of markers on a single surface. Particularly useful physical formats comprise surfaces having a plurality of discrete, adressable locations for the detection of a plurality of different analytes. Such formats include protein microarrays, or “protein chips” (see, for example, Ng and Ilag, J. Cell Mol. Med. 6: 329-340 (2002)) and certain capillary devices (see, e.g., U.S. Pat. No. 6,019,944). In these embodiments, each discrete surface location may comprise antibodies to immobilize one or more analyte(s) (e.g., a marker) for detection at each location. Surfaces may alternatively comprise one or more discrete particles (e.g., microparticles or nanoparticles) immobilized at discrete locations of a surface, where the microparticles comprise antibodies to immobilize one analyte (e.g., a marker) for detection.

In practice, the sensitivity and specificity of a marker for a particular diagnosis or prognosis is typically assessed using a “diseased” population and a “control” (e.g., a normal) population. While the terms “diseased” and “control” are used for convenience herein to refer to these populations, these terms refer to a first subject population exhibiting some characteristic of interest, and a second subject population not exhibiting that characteristic. The characteristic might be the presence or absence of a disease, a risk of some future outcome, etc. Receiver Operating Characteristic curves, or “ROC” curves, may be calculated by plotting the value of a variable versus its relative frequency in the “control” and “disease” populations. For any particular marker, a distribution of marker levels for subjects exhibiting and not exhibiting the characteristic of interest will likely overlap. Such a test need not absolutely distinguish “control” from “disease” with 100% accuracy, and the area of overlap indicates where the test cannot distinguish the control population from the disease population. A threshold value for the test is selected, above which (or below which, depending on how a marker changes with the disease) the test is considered to be indicative of one state or condition in a subject (e.g., disease, outcome, etc.) and below which the test is considered to be indicative of another state or condition in the subject. The area under the ROC curve is a measure of the probability that the perceived measurement will allow correct identification of a characteristic of interest. These methods are well known in the art. See, e.g., Hanley et al., Radiology 143: 29-36 (1982).

Measures of test accuracy may be obtained as described in Fischer et al., Intensive Care Med. 29: 1043-51, 2003; Zhou et al., Statistical Methods in Diagnostic Medicine, John Wiley & Sons, 2002; and Motulsky, Intuitive Biostatistics, Oxford University Press, 1995; and other publications well known to those of skill in the art, and used to determine the effectiveness of a given marker or panel of markers. These measures include sensitivity and specificity, predictive values, likelihood ratios, diagnostic odds ratios, hazard ratios, and ROC curve areas. As discussed above, suitable tests for detecting LRRTM1 polypeptides may exhibit one or more of the following results on these various measures.

In certain embodiments, a ROC curve area is greater than about 0.5, or greater than about 0.7, or greater than about 0.8, or greater than about 0.85, or greater than about 0.9; a positive or negative likelihood ratio of at least about 1.1 or more or about 0.91 or less, or at least about 1.25 or more or about 0.8 or less, or at least about 1.5 or more or about 0.67 or less, or at least about 2 or more or about 0.5 or less, or at least about 2.5 or more or about 0.4 or less; an odds ratio of at least about 2 or more or about 0.5 or less, or at least about 3 or more or about 0.33 or less, or at least about 4 or more or about 0.25 or less, or at least about 5 or more or about 0.2 or less, or at least about 10 or more or about 0.1 or less; and/or a hazard ratio of at least about 1.1 or more or about 0.91 or less, or at least about 1.25 or more or about 0.8 or less, or at least about 1.5 or more or about 0.67 or less, or at least about 2 or more or about 0.5 or less, or at least about 2.5 or more or about 0.4 or less.

Measures of diagnostic accuracy such as those discussed above are often reported together with confidence intervals or p values. These may be calculated by methods well known in the art. See, e.g., Dowdy and Wearden, Statistics for Research, John Wiley & Sons, New York, 1983. In certain embodiments, confidence intervals of the invention are 90%, or 95%, or 97.5%, or 98%, or 99%, or 99.5%, or 99.9%, or 99.99%. In certain embodiments, p values are 0.1, or 0.05, or 0.025, or 0.02, or 0.01, or 0.005, or 0.001, or 0.0001.

### Gene Expression of the Target Molecules in Cancer

Genes that are uniquely or differentially expressed in cancerous cells or tissues may potentially serve as cancer cell markers in bodily fluids, for example, serum. A reliable marker must be specific to cancer, and expressed only when the patient has cancer. The results of the bioinformatics, microarray hybridization, and quantitative PCR studies presented herein demonstrate that LRRTM1 is a cancer cell marker useful for diagnosing cancer, for example ovarian cancer, pancreatic cancer, and colon/colorectal cancer in body fluid samples.

### Active Agents (or Modulators)

The nucleic acid, polypeptide, and modulator compositions of the subject invention find use as therapeutic agents in situations where one wishes to modulate LRRTM1 activity, or to provide or inhibit LRRTM1 activity at a particular anatomical site. The active agents of the invention are useful in the diagnosis and treatment of proliferative diseases, for example, ovarian cancer, pancreatic cancer, and colon/colorectal cancer. Modulators of the invention include, for example, polypeptide variants, whether agonist or antagonist; aptamers, antibodies, whether agonist or antagonist, interfering or specific; soluble receptors, usually antagonists; small molecule drugs, whether agonist or antagonist; RNAi, usually an antagonist; antisense molecules, usually antagonists; and ribozymes, usually antagonists.

In an embodiment, modulators of the invention bind to target LRRTM1 molecules. They may directly inhibit LRRTM1 as a result of their binding. They may also indirectly modulate a biological process by interacting with LRRTM1. Modulators of the invention may bind to LRRTM1 in a manner that may or may not interfere with the function of the target LRRTM1 molecule; the modulator may be therapeutically efficacious whether or not the modulator interferes with LRRTM1 function. For example, a modulator may form a complex with LRRTM1 and an effector molecule or effector cell.

In some embodiments, an agent is an LRRTM1 polypeptide, where the LRRTM1 polypeptide itself is administered to an individual. In some embodiments, an agent is an antibody specific for a subject target polypeptide. In some embodiments, an agent is a chemical compound, such as a small molecule, that may be useful as an orally available drug. Such modulation may include the recruitment of other molecules that directly effect the modulation. For example, an antibody that modulates the activity of a subject polypeptide that is a receptor on a cell surface may bind to the receptor and fix complement, activating the complement cascade and result in lysis of the cell. An agent which modulates a biological activity of a subject polypeptide or polynucleotide increases or decreases the activity or binding at least about 10%, at least about 15%, at least about 20%, at least about 25%, at least about 50%, at least about 100%, or at least about 2-fold, at least about 5-fold, or at least about 10-fold or more when compared to a suitable control.

The invention provides a method of identifying a modulator of the biological activity of a polypeptide of the invention by providing at least one polypeptide chosen from the sequences listed in the Tables, Figures, and Sequence Listing, and active fragments thereof; allowing at least one agent to contact the polypeptide; and selecting an agent that binds the polypeptide or affects the biological activity of the polypeptide. In an embodiment, the modulator is an antibody.

The invention provides compositions comprising modulators obtained by this method and a pharmaceutically acceptable carrier. For example, the invention provides modulator compositions comprising a pharmaceutically acceptable carrier and a modulator, wherein the modulator is a soluble receptor that competes for ligand binding or cofactor binding to an isolated polypeptide comprising an amino acid sequence chosen from the Tables, Figures, and Sequence Listing, and biologically active fragments thereof. The invention also provides a modulator composition comprising a pharmaceutically acceptable carrier and a modulator, wherein the modulator is an extracellular fragment that competes for ligand binding or cofactor binding to an isolated polypeptide comprising an amino acid sequence chosen from the Tables, Figures, and Sequence Listing, and biologically active fragments thereof.

Antisense Oligonucleotides

In certain embodiments of the invention, the agent is an antisense molecule that modulates, and generally decreases or down regulates, polypeptide expression in a host (Agrawal, S., Crooke, S. T. eds. (1998) Antisense Research and Application (Handbook of Experimental Pharmacology, Vol 131), Springer-Verlag New York, Inc.; Hartmann, G., et al., eds. (1999) Manual of Antisense Methodology (Perspectives in Antisense Science. 1^{st} ed. Kluwer Law International; Phillips, M. I., ed. (1999a) Antisense Technology, Part A. Methods in Enzymology Vol. 313. Academic Press, Inc.; Phillips, M. I., ed. (1999b) Antisense Technology, Part B. Methods in Enzymology Vol. 314, Academic Press, Inc.; Stein, C. A., et al., eds. (1998) Applied Antisense Oligonucleotide Technology. Wiley-Liss). Accordingly, the invention provides a modulator composition comprising a pharmaceutically acceptable carrier and a modulator, wherein the modulator is an antisense molecule that inhibits the transcription or translation of an isolated polynucleotide or an isolated polypeptide comprising an amino acid sequence encoded by a polynucleotide chosen from the Tables, Figures, and Sequence Listing, and biologically active fragments thereof. The invention also provides a modulator composition comprising a pharmaceutically acceptable carrier and a modulator, wherein the modulator is a ribozyme that inhibits the transcription or translation of an isolated polynucleotide or an isolated polypeptide comprising an amino acid sequence encoded by a polynucleotide chosen from the Tables, Figures, and Sequence Listing, and biologically active fragments thereof.

Antisense reagents of the invention include antisense oligonucleotides (ODN), for example, synthetic ODN having chemical modifications from native nucleic acids, or nucleic acid constructs that express such antisense molecules as RNA. The antisense sequence is complementary to the mRNA of the targeted LRRTM1 gene, and inhibits expression of the targeted LRRTM1 gene products. Antisense molecules inhibit LRRTM1 gene expression through various mechanisms, for example, by reducing the amount of mRNA available for translation, through activation of RNase H, or steric hindrance. One or a combination of antisense molecules can be administered, where a combination can comprise multiple different sequences.

Antisense molecules can be produced by expression of all or a part of the LRRTM1 gene sequence in an appropriate vector, where the transcriptional initiation is oriented such that an antisense strand is produced as an RNA molecule. Alternatively, the antisense molecule is a synthetic oligonucleotide. Antisense oligonucleotides can be chemically synthesized by methods known in the art (Wagner, R. W., et al. (1993) Science 260:1510-1513; Milligan, J. F., et al. (1993) J. Med. Chem. 36:1923-1937). Antisense oligonucleotides will generally be at least about seven, at least about 12, or at least about 20 nucleotides in length, and not more than about 500, not more than about 50, or not more than about 35 nucleotides in length, where the length is governed by efficiency of inhibition, and specificity, including absence of cross-reactivity, and the like. Short oligonucleotides, of from about seven to about eight bases in length, can be strong and selective inhibitors of gene expression (Wagner, R. W., et al. (1996) Nat. Biotechnol. 14:840-844).

A specific region or regions of the endogenous sense strand LRRTM1 mRNA sequence is chosen to be complemented by the antisense sequence. Selection of a specific sequence for the oligonucleotide can use an empirical method, where several candidate sequences are assayed for inhibition of expression of the target gene in an in vitro or animal model. As noted above, a combination of sequences can also be used, where several regions of the mRNA sequence are chosen for antisense complementation.

As an alternative to antisense inhibitors, catalytic nucleic acid compounds, for example, ribozymes, or antisense conjugates can be used to inhibit gene expression. Ribozymes can be synthesized in vitro and administered to the patient, or can be encoded in an expression vector, from which the ribozyme is synthesized in the targeted cell (WO 9523225; Beigelman, L., et al. (1995) Nucleic Acids Res. 23:4434-4442). Examples of oligonucleotides with catalytic activity are described in WO 9506764. Conjugates of antisense ODN with a metal complex, for example, terpyridyl Cu(II), capable of mediating mRNA hydrolysis are described in Bashkin, J. K., et al. (1995) Appl. Biochem. Biotechnol. 54:43-56.

Interfering RNA

In some embodiments, the active agent is an interfering RNA (RNAi). RNA interference provides a method of silencing eukaryotic genes. Use of RNAi to reduce a level of a particular mRNA and/or protein is based on the interfering properties of RNA, for example, double-stranded RNA (dsRNA), derived from the coding regions of a gene. The technique is an efficient high-throughput method for disrupting gene function (O'Neil, N. J., et al., (2001) Am. J. Pharmacogenomics 1:45-53). RNAi can also help identify the biochemical mode of action of a drug and to identify other genes encoding products that can respond or interact with specific compounds. Accordingly, the invention provides a modulator composition comprising a pharmaceutically acceptable carrier and a modulator, wherein the modulator is an RNAi molecule that inhibits the transcription or translation of an isolated polynucleotide or an isolated polypeptide comprising an amino acid sequence encoded by a polynucleotide chosen from the Tables, Figures, and Sequence Listing, and biologically active fragments thereof.

In one embodiment of the invention, complementary sense and antisense RNAs derived from a substantial portion of a polynucleotide encoding LRRTM1 are synthesized in vitro. The resulting sense and antisense RNAs are annealed in an injection buffer, and the double-stranded RNA injected or otherwise introduced into the subject, for example, in food or by immersion in buffer containing the RNA (Gaudilliere, B., et al. (2002) J. Biol. Chem. 277:46,442-46,446; O'Neil et al., 2001; WO99/32619). In an embodiment, dsRNA derived from an LRRTM1 gene is generated in vivo by simultaneously expressing both sense and antisense RNA from appropriately positioned promoters operably linked to coding sequences in both sense and antisense orientations.

Aptamers

Another suitable agent for modulating an activity of a subject polypeptide is an aptamer. Aptamers of the invention include both nucleotide and peptide aptamers. Nucleotide aptamers of the invention include double stranded DNA and single stranded RNA molecules that bind to LRRTM1 proteins or fragments thereof. Peptide aptamers are peptides or small polypeptides that act as dominant inhibitors of protein function. Peptide aptamers specifically bind to target proteins, blocking their functional ability (Kolonin, M. G., et al. (1998) Proc. Natl. Acad. Sci. 95:14,266-14,271). Due to the highly selective nature of peptide aptamers, they can be used not only to target a specific protein, but also to target specific functions of a given protein (for example, a signaling function). Further, peptide aptamers can be expressed in a controlled fashion by use of promoters which regulate expression in a temporal, spatial, or inducible manner. Peptide aptamers act dominantly, therefore, they can be used to analyze proteins for which loss-of-function mutants are not available. Aptamers of the invention may bind nucleotide cofactors (Latham, J. A., et al. (1994) Nucl. Acids Res. 22:2817-2822).

Peptide aptamers that bind with high affinity and specificity to a target protein can be isolated by a variety of techniques known in the art. Peptide aptamers can be isolated from random peptide libraries by yeast two-hybrid screens (Xu, C. W., et al. (1997) Proc. Natl. Acad. Sci. 94:12,473-12,478). They can also be isolated from phage libraries (Hoogenboom, H. R., et al. (1998) Immunotechnology 4:1-20) or chemically generated peptides/libraries.

Peptides and Modified Peptides

Polypeptides of the invention include full length proteins that include a signal peptide or leader sequence, if present, or a mature protein from which a signal peptide or leader sequence may have been cleaved, the signal peptide or leader sequence, or portions or fragments of the full length or mature protein. Also included in this term are biologically active variations of naturally occurring proteins, where such variations are homologous or substantially similar to the naturally occurring protein, as well as corresponding homologs from different species. Variants of polypeptide sequences may include insertions, additions, deletions, or substitutions compared with the subject polypeptides. Variants of polypeptide sequences include biologically active polymorphic variants.

In some embodiments of the present invention, the active agent is a peptide. Suitable peptides include peptides of from about three amino acids to about 50, from about five to about 30, or from about 10 to about 25 amino acids in length which may, but need not, correspond to the sequence of the naturally-occurring protein. In some embodiments, a peptide has a sequence of from about seven amino acids to about 45, from about nine to about 35, or from about 12 to about 25 amino acids of corresponding naturally-occurring protein. In some embodiments, a peptide exhibits one or more of the following activities: inhibits binding of a subject polypeptide to an interacting protein or other molecule; inhibits subject polypeptide binding to a second polypeptide molecule; inhibits a signal transduction activity of a subject polypeptide; inhibits an enzymatic activity of a subject polypeptide; or inhibits a DNA binding activity of a subject polypeptide.

Peptides of the invention can include naturally-occurring and non-naturally occurring amino acids. Peptides can comprise D-amino acids, a combination of D- and L-amino acids, and various “designer” or “synthetic” amino acids (for example, β-methyl amino acids, Cα-methyl amino acids, and Nα-methyl amino acids, etc.) to convey special properties. Additionally, peptides can be cyclic. Peptides can include non-classical amino acids in order to introduce particular conformational motifs. Any known non-classical amino acid can be used. Non-classical amino acids include, but are not limited to, 1,2,3,4-tetrahydroisoquinoline-3-carboxylate; (2S,3S)-methylphenylalanine, (2S,3R)-methyl-phenylalanine, (2R,3S)-methyl-phenylalanine and (2R,3R)-methyl-phenylalanine; 2-iminotetrahydro-naphthalene-2-carboxylic acid; hydroxy-1,2,3,4-tetrahydroisoquinoline-3-carboxylate; β-carboline (D and L); HIC (histidine isoquinoline carboxylic acid); and HIC (histidine cyclic urea). Amino acid analogs and peptidomimetics can be incorporated into a peptide to induce or favor specific secondary structures, including, but not limited to, LL-Acp (LL-3-amino-2-propenidone-6-carboxylic acid), a β-turn inducing dipeptide analog; β-sheet inducing analogs; β-turn inducing analogs; α-helix inducing analogs; γ-turn inducing analogs; Gly-Ala turn analogs; amide bond isostere; or tretrazol, and the like.

An LRRTM1 peptide of the invention can be a depsipeptide, which can be linear or cyclic (Kuisle, O., et al., (1999) Tetrahedron Lett. 40:1203-1206). Linear depsipeptides can comprise rings formed through S—S bridges, or through an hydroxy or a mercapto group of an hydroxy-, or mercapto-amino acid and the carboxyl group of another amino- or hydroxy-acid but do not comprise rings formed only through peptide or ester links derived from hydroxy carboxylic acids. Cyclic depsipeptides contain at least one ring formed only through peptide or ester links, derived from hydroxy carboxylic acids.

LRRTM1 peptides of the invention can be monocyclic or bicyclic. For example, the C-terminal carboxyl group or a C-terminal ester can be induced to cyclize by internal displacement of the (—OH) or the ester (—OR) of the carboxyl group or ester respectively with the N-terminal amino group to form a cyclic peptide. For example, after synthesis and cleavage to give the peptide acid, the free acid is converted to an activated ester by an appropriate carboxyl group activator such as dicyclohexylcarbodiimide (DCC) in solution, for example, in methylene chloride (CH₂Cl₂), dimethyl formamide (DMF) mixtures. The cyclic peptide is then formed by internal displacement of the activated ester with the N-terminal amine. Internal cyclization as opposed to polymerization can be enhanced by use of very dilute solutions. Methods for making cyclic peptides are well known in the art.

A desamino or descarboxy residue can be incorporated at the terminal ends of the peptide, so that there is no terminal amino or carboxyl group, to decrease susceptibility to proteases or to restrict conformation. C-terminal functional groups include amide, amide lower alkyl, amide di (lower alkyl), lower alkoxy, hydroxy, and carboxy, and the lower ester derivatives thereof, and the pharmaceutically acceptable salts thereof.

In addition to the foregoing N-terminal and C-terminal modifications, an LRRTM1 peptide or peptidomimetic can be modified with or covalently coupled to one or more of a variety of hydrophilic polymers to increase solubility and circulation half-life of the peptide. Suitable nonproteinaceous hydrophilic polymers for coupling to a peptide include, but are not limited to, polyalkylethers as exemplified by polyethylene glycol and polypropylene glycol, polylactic acid, polyglycolic acid, polyoxyalkenes, polyvinylalcohol, polyvinylpyrrolidone, cellulose and cellulose derivatives, dextran, and dextran derivatives. Generally, such hydrophilic polymers have an average molecular weight ranging from about 500 to about 100,000 daltons, from about 2,000 to about 40,000 daltons, or from about 5,000 to about 20,000 daltons. The peptide can be derivatized with or coupled to such polymers using any of the methods set forth in Zallipsky, S. (1995) Bioconjugate Chem., 6:150-165; Monfardini, C., et al. (1995) Bioconjugate Chem. 6:62-69; U.S. Pat. Nos. 4,640,835; 4,496,689; 4,301,144; 4,670,417; 4,791,192; 4,179,337, or WO 95/34326.

Small Molecules

Small molecule modulators such as those commonly used as therapeutic drugs, can be used as LRRTM1 in the invention. Small molecule agents include chemical compounds that bind the LRRTM1 polypeptide and inhibit an activity of the polypeptide or a cell containing the polypeptide. Small molecule inhibitors of the invention may permeate the cell, and/or may exert their action at the extracellular surface or on non-cellular structures, such as the extracellular matrix.

Antibodies

Modulators of the invention may be antibodies. The invention provides an isolated antibody that specifically recognizes, binds to, interferes with, and/or otherwise modulates the biological activity of at least one LRRTM1 polypeptide of the Tables, Figures, and Sequence Listing or a polypeptide encoded by an LRRTM1 nucleic acid molecule of the Tables, Figures, and Sequence Listing. An antibody modulator of a polypeptide is a modulator that recognizes and binds specifically to the polypeptide. Such an antibody may, for example, induce ADCC, CDC, or apoptosis, or may block or otherwise interfere with the activity of an LRRTM1 polypeptide.

In addition, an antibody of the invention may be directed to a polypeptide comprising a non-transmembrane domain and/or an extracellular domain. A non-transmembrane domain is a portion of a transmembrane protein that does not span the membrane. It may be extracellular, cytoplasmic, or luminal. An antibody of the invention may be directed to polypeptide comprising a part or all of a Pfam domain, signal peptide, propeptide, N-terminal or C-terminal domain, LRR domain, or cytoplasmic tail of LRRTM1.

The production and use of antibodies is well-known in the art (Harlow, E., et al., eds. (1998) Using Antibodies: A Laboratory Manual: Portable Protocol NO. I. Cold Spring Harbor Laboratory; Harlow, E., Lane, D., eds. (1988) Antibodies: A Laboratory Manual. Cold Spring Harbor Laboratory; Howard, G. C., et al. (2000) Basic Methods in Antibody Production and Characterization, CRC Press). This antibody may be a monoclonal antibody; a polyclonal antibody; a single chain antibody; an antibody comprising a backbone of a molecule with an Ig domain or a T cell receptor backbone; a targeting antibody; a neutralizing antibody; a stabilizing antibody; an enhancing antibody; an antibody agonist; an antibody antagonist; an antibody that promotes endocytosis of a target antigen; a cytotoxic antibody; an antibody that mediates antibody-dependent cell cytotoxicity, a human antibody; a non-human primate antibody; a non-primate animal antibody; or an antibody that mediates complement-dependent cytotoxicity.

An antibody of the invention can be a human antibody, a non-human primate antibody, a non-primate animal antibody, a rabbit antibody, a mouse antibody, a rat antibody, a sheep antibody, a goat antibody, a horse antibody, a porcine antibody, a cow antibody, a chicken antibody, a humanized antibody, a primatized antibody, and/or a chimeric antibody. Antibodies of the invention can comprise a cytotoxic antibody with one or more cytotoxic component chosen from a radioisotope, a microbial toxin, a plant toxin, and a chemical compound. The chemical compound can, for example, be chosen from doxorubicin and cisplatin. Antibodies of the invention include antigen-binding fragments; fragments comprising a variable region of a heavy chain or a light chain of an immunoglobulin; fragments comprising a complementarity determining region or a framework region of an immunoglobulin; and one or more active fragments, analogues, and/or antagonists.

The isolated antibodies of the invention can be produced in a variety of cells. Host cells of the invention can be genetically modified to produce an antibody of the invention; these include bacterial cells, fungal cells, plant cells, insect cells, and mammalian cells. For example, isolated antibodies of the invention may be produced in yeast cells, Aspergillus cells, SF9 cells, High Five cells, cereal plant cells, tobacco cells, tomato cells, human kidney embryonic kidney 293 cells, myeloma cells, including mouse myeloma NS0 cells, human fetal Per C6 cells, and CHO cells.

In another aspect, the invention provides antibody targets. The LRRTM1 polynucleotides and polypeptides described herein comprise nucleic acid and amino acid sequences that can be recognized by antibodies. A target sequence can be any polynucleotide or amino acid sequence of approximately eighteen or more contiguous nucleotides or approximately six or more amino acids. A variety of comparing means can be used to accomplish comparison of sequence information from a sample (for example, to analyze target sequences, target motifs, or relative expression levels) with the data storage means. A skilled artisan can readily recognize that any one of the publicly available homology search programs can be used as the search means for the computer based systems of the present invention to accomplish comparison of target sequences and motifs. Computer programs to analyze expression levels in a sample and in controls are also known in the art. A target sequence includes an antibody target sequence, which refers to an amino acid sequence that can be used as an immunogen for injection into animals for production of antibodies or for screening against a phage display or antibody library for identification of binding partners.

The invention provides target structural motifs and target functional motifs, i.e., any rationally selected sequences or combination of sequences in which the sequence(s) are chosen based on a three-dimensional configuration formed upon the folding of the target motif, or on consensus sequences of regulatory or active sites. There are a variety of target motifs known in the art. Protein target motifs include, but are not limited to, enzyme active sites and signal sequences. Nucleic acid target motifs include, but are not limited to, hairpin structures, promoter sequences, and other expression elements, such as binding sites for transcription factors.

Antibodies of the invention bind specifically to their targets. Specific binding, in the context of antibody binding, refers to high avidity and/or high affinity binding of an antibody to a specific polypeptide, or more accurately, to an epitope of a specific polypeptide. Antibody binding to such an epitope on a polypeptide can be stronger than binding of the same antibody to any other epitopes, particularly other epitopes that can be present in molecules in association with, or in the same sample as the polypeptide of interest. For example, when an antibody binds more strongly to one epitope than to another, adjusting the binding conditions can result in antibody binding almost exclusively to the specific epitope and not to any other epitopes on the same polypeptide, and not to any other polypeptide, which does not comprise the epitope. Antibodies that bind specifically to a subject polypeptide may be capable of binding other polypeptides at a weak, yet detectable, level (for example, 10% or less of the binding shown to the polypeptide of interest). Such weak binding, or background binding, is readily discernible from the specific antibody binding to a subject polypeptide, for example, by use of appropriate controls. In general, antibodies of the invention bind to a specific polypeptide with a binding affinity of 10⁷ M⁻¹ or greater (for example, 10⁸ M⁻¹, 10⁹ M⁻¹, 10¹⁰ M⁻¹, 10¹¹ M⁻¹, etc.).

The invention provides antibodies that can distinguish variant LRRTM1 sequences from one another. These antibodies can distinguish polypeptides that differ by no more than one amino acid (U.S. Pat. No. 6,656,467). They have high affinity constants, which are in the range of approximately 10¹⁰ M⁻¹, and are produced, for example, by genetically engineering appropriate antibody gene sequences, according to the method described by Young et al., in U.S. Pat. No. 6,656,467.

Antibodies of the invention can be provided as matrices, for example, as geometric networks of antibody molecules and their antigens, as found in immunoprecipitation and flocculation reactions. An antibody matrix can exist in solution or on a solid phase support.

Antibodies of the invention can be provided as a library of antibodies or fragments thereof, wherein at least one antibody or fragment thereof specifically binds to at least a portion of a polypeptide comprising an amino acid sequence or fragment thereof described in the Tables or Sequence Listing, and/or wherein at least one antibody or fragment thereof interferes with at least one activity of the polypeptide or fragment thereof. In certain embodiments, the antibody library comprises at least one antibody or fragment thereof that specifically inhibits the binding of an LRRTM1 polypeptide to its ligand or substrate, or that specifically inhibits binding of an LRRTM1 polypeptide as a substrate to another molecule. In certain embodiments, the antibody library comprises combinatorial complementarity determining regions, heavy chains, and light chains. The present invention also features corresponding polynucleotide libraries comprising at least one polynucleotide sequence that encodes an antibody or antibody fragment of the invention. In specific embodiments, the library is provided on a nucleic acid array or in computer-readable format.

The invention provides a method of making an antibody by introducing an antigen chosen from an isolated nucleic acid molecule comprising at least one polynucleotide sequence chosen from the Tables or Sequence Listing; sequences that hybridize to these sequences under high stringency conditions; sequences having at least 80% sequence identity to these sequences, or sequences that hybridize to them under high stringency conditions; complements of any of these sequences; or biologically active fragments of any of the above-listed sequences or an isolated polypeptide comprising an amino acid sequence, wherein the amino acid sequence is chosen from the Tables, Figures, or Sequence Listing, or a biologically active fragment thereof, or is encoded by a polynucleotide sequence chosen from the Tables, Figures, or Sequence Listing, or a biologically active fragment thereof, into an animal in an amount sufficient to elicit generation of antibodies specific to the antigen, and recovering the antibodies therefrom.

The immunogen can comprise a nucleic acid, a complete protein, or fragments and derivatives thereof, or proteins expressed on cell surfaces. Protein domains, for example, Pfam domains, or extracellular, cytoplasmic, or luminal domains can be used as immunogens. Immunogens can comprise all or a part of one of the LRRTM1 proteins, where these amino acids contain post-translational modifications, such as glycosylation, found on the native target protein. Immunogens comprising protein extracellular domains are produced in a variety of ways known in the art, for example, expression of cloned genes using conventional recombinant methods, or isolation from tumor cell culture supernatants, etc. The immunogen can also be expressed in vivo from a polynucleotide encoding the immunogenic peptide introduced into the host animal.

Polyclonal antibodies of the invention are prepared by conventional techniques. These include immunizing the host animal in vivo with the target protein (or immunogen) in substantially pure form, for example, comprising less than about 1% contaminant. The immunogen can comprise the complete target protein, fragments, or derivatives thereof. To increase the immune response of the host animal, the target protein can be combined with an adjuvant; suitable adjuvants include alum, dextran, sulfate, large polymeric anions, and oil and water emulsions, for example, Freund's adjuvant (complete or incomplete). The target protein can also be conjugated to synthetic carrier proteins or synthetic antigens. The target protein is administered to the host, usually intradermally, with an initial dosage followed by one or more, usually at least two, additional booster dosages. Following immunization, blood from the host is collected, followed by separation of the serum from blood cells. The immunoglobulin present in the resultant antiserum can be further fractionated using known methods, such as ammonium salt fractionation, or DEAE chromatography and the like.

Monoclonal antibodies of the invention are also produced by conventional techniques, such as fusing an antibody-producing plasma cell with an immortal cell to produce hybridomas. Suitable animals will be used, for example, mice. Also by way of example, to raise antibodies against a mouse polypeptide of the invention, the host animal will generally be a hamster, guinea pig, goat, chicken, rabbit, or the like. Generally, the spleen and/or lymph nodes of an immunized host animal provide the source of plasma cells, which are immortalized by fusion with myeloma cells to produce hybridoma cells. Culture supernatants from individual hybridomas are screened using standard techniques to identify clones producing antibodies with the desired specificity. The antibody can be purified from the hybridoma cell supernatants or from ascites fluid present in the host by conventional techniques, for example, affinity chromatography using antigen, for example, the subject protein, bound to an insoluble support, for example, protein A Sepharose®, etc.

Cytokines can be used to help stimulate immune response. Cytokines act as chemical messengers, stimulating optimal responses from immune cells. An example of a cytokine is granulocyte-macrophage colony-stimulating factor (GM-CSF), which stimulates the proliferation of antigen-presenting cells, thus boosting an organism's response to a cancer vaccine. As with adjuvants, cytokines can be used in conjunction with the antibodies and vaccines disclosed herein. For example, they can be incorporated into the antigen-encoding plasmid or introduced via a separate plasmid, and in some embodiments, a viral vector can be engineered to display cytokines on its surface.

The antibody can be produced as a single chain, instead of the normal multimeric structure of the immunoglobulin molecule. Single chain antibodies have been previously described by Jost, C. R., et al. (1994) J. Biol. Chem. 269:26,267-26,273. DNA sequences encoding parts of the immunoglobulin, for example, the variable region of the heavy chain and the variable region of the light chain are ligated to a spacer, such as one encoding at least about four small neutral amino acids, such as glycine or serine. The protein encoded by this fusion allows the assembly of a functional variable region that retains the specificity and affinity of the original antibody.

The invention also provides intrabodies that are intracellularly expressed single-chain antibody molecules designed to specifically bind and inactivate target molecules inside cells. Intrabodies have been used in cell assays and in whole organisms (Chen, S. Y., et al. (1994) Hum. Gene Ther. 5:595-601; Hassanzadeh, G. H. G., et al. (1998) FEBS Lett. 437:75-80). Inducible expression vectors can be constructed with intrabodies that react specifically with a protein of the invention. These vectors can be introduced into host cells and model organisms.

The invention provides artificial antibodies, which are fragments produced and selected in vitro. In some embodiments, these antibodies, or fragments thereof, are displayed on the surface of a bacteriophage or other viral particle, as described above. Suitable fragments include single chain variable region antibodies. In other embodiments, artificial antibodies are present as fusion proteins with a viral or bacteriophage structural protein, including, but not limited to, M13 gene III protein. Methods of producing such artificial antibodies are well known in the art (U.S. Pat. Nos. 5,516,637; 5,223,409; 5,658,727; 5,667,988; 5,498,538; 5,403,484; 5,571,698; and 5,625,033). The artificial antibodies, selected, for example, on the basis of phage binding to selected antigens, can be fused to a Fc fragment of an immunoglobulin for use as a therapeutic, as described, for example, in U.S. Pat. No. 5,116,964 or WO 99/61630.

In an embodiment, artificial antibodies of the invention include genetically engineered antibodies. Single chain variable region antibodies are within the scope of such an embodiment. Engineered antibodies may incorporate non-antibody domains, including, for example, coiled coil domains for dimerization, linkers, or other such useful modifications. Genetically engineered antibodies of the invention include proteins with predetermined ligand specificity based on a known or predicted epitope, for example anticalins (Schlehuber, S., et al. (2001) Biol. Chem. 382:1335-1342), which are suitable for use in the invention when an immunogenic, cross-linking, or effector property of an antibody is undesirable.

For in vivo use, particularly for injection into humans, in some embodiments it is desirable to decrease the antigenicity of the antibody. An immune response of a recipient against the antibody may potentially decrease the period of time that the therapy is effective. Methods of humanizing antibodies are known in the art. The humanized antibody, for example, a fully human antibody, can be the product of an animal having transgenic human immunoglobulin genes, for example, constant region genes (for example, Grosveld, F., Kollias, G., eds. (1992) Transgenic Animals. 1^{st} ed. Academic Press; Murphy, D., et al., eds. (1993) Transgenesis Techniques: Principles and Protocols. Humana Press; Pinkert, C. A., ed. (1994) Transgenic Animal Technology: A Laboratory Handbook, Academic Press; and International Patent Applications WO 90/10077 and WO 90/04036). Alternatively, the antibody of interest can be engineered by recombinant DNA techniques to substitute the CH1, CH2, CH3, hinge domains, and/or the framework domain with the corresponding human sequence (see, for example, WO 92/02190).

Thus, antibodies of the invention can be partially human or fully human antibodies. For example, xenogenic antibodies, which are produced in animals that are transgenic for human antibody genes, can be employed to make a fully human antibody. By xenogenic human antibodies is meant antibodies that are fully human antibodies, with the exception that they are produced in a non-human host that has been genetically engineered to express human antibodies (for example, WO 98/50433; WO 98/24893 and WO 99/53049).

Humanized antibodies can be produced by immunizing mice that make human antibodies. Abgenix's XenoMouse (for example, U.S. Pat. Nos. 5,939,598; 6,075,181; 6,091,001; 6,114,598; 6,150,584; 6,162,963; 6,657,103; 6,673,986; 6,682,736) Medarex's mice (for example, U.S. Pat. Nos. 5,922,845; 6,111,166; 6,410,690; 6,680,209) and Kirin's mice (for example, U.S. Pat. Nos. 6,320,099; 6,632,976) are suitable for use in the invention. Humanized antibodies can be made, for example, using the technology of Protein Design Labs, Inc. (Fremont, Calif.) (for example, Coligan, J. E. et al., eds. (2002) Current Protocols in Immunology, vols. 1-4, including suppl.) John Wiley and Sons, Inc. New York, N.Y.). Both polyclonal and monoclonal antibodies made in non-human animals may be humanized before administration to human subjects.

Chimeric immunoglobulin genes constructed with immunoglobulin cDNA are known in the art (Liu A. Y., et al. (1987a) Proc. Natl. Acad. Sci. 84:3439-3443; Liu, A. Y., et al. (1987b) J. Immunol. 139:3521-26.). Messenger RNA is isolated from a hybridoma or other cell producing the antibody and used to produce cDNA. The cDNA of interest can be amplified by the polymerase chain reaction using specific primers (U.S. Pat. Nos. 4,683,195 and 4,683,202). Alternatively, a library is made and screened to isolate the sequence of interest. The DNA sequence encoding the variable region of the antibody is then fused to human constant region sequences. The sequences of human constant (C) regions genes are known in the art (Kabat, E. A., Wu T. T. (1991) J. Immunol. 147:1709-1719). Human C region genes are readily available from known clones. The choice of isotype will be guided by the desired effector functions, such as complement fixation, or antibody-dependent cellular cytotoxicity. IgG1, IgG2, IgG3, and IgG4 isotypes, and either of the kappa or lambda human light chain constant regions can be used. The chimeric, humanized antibody is then expressed by conventional methods.

Consensus sequences of heavy (H) and light (L) J regions can be used to design oligonucleotides for use as primers to introduce useful restriction sites into the J region for subsequent linkage of V region segments to human C region segments. C region cDNA can be modified by site-directed mutagenesis to place a restriction site at the analogous position in the human sequence.

A convenient expression vector for producing antibodies is one that encodes a functionally complete human CH or CL immunoglobulin sequence, with appropriate restriction sites engineered so that any VH or VL sequence can be easily inserted and expressed, such as plasmids, retroviruses, YACs, or EBV-derived episomes, and the like. In such vectors, splicing usually occurs between the splice donor site in the inserted J region and the splice acceptor site preceding the human C region, and also at the splice regions that occur within the human CH exons. Polyadenylation and transcription termination occur at native chromosomal sites downstream of the coding regions. The resulting chimeric antibody can be joined to any strong promoter, including retroviral LTRs, for example, SV-40 early promoter (Okayama, H., et al. (1983) Mol. Cell. Biol. 3:280-289), Rous sarcoma virus LTR (Gorman, C. M., et al. (1982) Proc. Natl. Acad. Sci. 79:6777-6781), and Moloney murine leukemia virus LTR (Grosschedl, R., Baltimore, D. (1985) Cell 41:885-897), or native immunoglobulin promoters.

Antibody fragments, such as Fv, F(ab′)₂, and Fab can be prepared by cleavage of the intact protein, for example, by protease or chemical cleavage. These fragments can include heavy and light chain variable regions. Alternatively, a truncated gene can be designed, for example, a chimeric gene encoding a portion of the F(ab′)₂ fragment that includes DNA sequences encoding the CH1 domain and hinge region of the H chain, followed by a translational stop codon.

Antibodies may be administered by injection systemically, such as by intravenous injection; or by injection or application to the relevant site, such as by direct injection into a tumor, or direct application to the site when the site is exposed in surgery, or by topical application, such as if the disorder is on the skin, for example.

The antibodies of the present invention may be administered alone or in combination with other molecules for use as a therapeutic, for example, by linking the antibody to cytotoxic agents or radioactive molecules. Radioactive antibodies and antibodies comprising a cytotoxic microbial, plant, or chemical compound that are specific to a cancer cell, diseased cell, or other target cell may be able to deliver a sufficient dose of radioactivity or toxin to kill the cell.

Radiolabeled antibodies of the invention can be used clinically to detect tumor cells, including latent metastases. Radionuclide imaging can be performed according to well-known methods, including that described in Kufe et al., eds. (2003) Cancer Medicine 6th ed., B. C. Decker, Inc. In vivo diagnostic imaging methods of the invention include single photon and positron imaging, and may include the use of scanners and cameras, including, but not limited to computed tomography (CT) scanners and gamma cameras.

Antibodies of the invention can be used to modulate biological activity of cells, either directly or indirectly. An LRRTM1 antibody can modulate the activity of a target cell, with which it has primary interaction, or it can modulate the activity of other cells by exerting secondary effects, for example in instances when the primary targets interact or communicate with other cells. An LRRTM1 antibody can also modulate the activity of a target cell by primarily interacting with an antigen, which then exerts an effect, whether direct, or indirect, on a target cell. Antibodies of the invention may specifically inhibit the binding of an LRRTM1 polypeptide to a ligand, specifically inhibit the binding of an LRRTM1 polypeptide to a substrate, specifically inhibit the binding of an LRRTM1 polypeptide as a ligand, specifically inhibit the binding of an LRRTM1 polypeptide as a substrate, specifically inhibit cofactor binding, induce apoptosis, induce ADCC, induce CDC, inhibit protease activity, inhibit adhesion, inhibit ligand/receptor interaction, and/or inhibit enzyme/substrate interaction.

The invention provides a method of modulating the biological activity of a first human or non-human animal host cell by providing an antibody of the invention and contacting the antibody with the first host cell, wherein the activity of the first host cell, and/or a second host cell, is modulated. For example, the first host cell may be a cancer cell and the second host cell may be a lymphocyte, NK cell, granulocyte, neutrophil, eosinophil, basophil, mast cell, macrophage, dendritic cell, or antigen presenting cell. In an embodiment, the first host cell expresses a polypeptide and the second host cell is an effector. In an embodiment, the antibody modulator first binds to the first host cell. In an embodiment, the antibody modulator first binds to the second host cell. In an embodiment, contacting the antibody with the first host cell results in recruitment of at least one second host cell.

This method of modulation includes embodiments wherein the modulation of biological activity is chosen from inhibiting cell activity directly, inhibiting cell activity indirectly, inducing antibody-dependent cell cytotoxicity, and inducing complement-dependent cytotoxicity. The modulated cell activity can include signal transduction, transcription, and translation. The modulated activity may be cell mobility, cell metastasis, cell invasion, and/or cell adhesion. The modulation of cell activity may result in cell death and/or inhibition of cell growth.

The antibodies of the invention can be administered to mammals, and the present invention includes such administration, for example, for therapeutic and/or diagnostic purposes in humans. Accordingly, the invention provides a pharmaceutical composition comprising a pharmaceutically acceptable carrier and an antibody of the invention.

The antibodies of the present invention can also be used in assays to detect LRRTM1 polypeptides. In some embodiments, the assay is a binding assay that detects binding of a polypeptide with an antibody specific for the polypeptide; the subject polypeptide or antibody can be immobilized, while the subject polypeptide and/or antibody can be detectably labeled. For example, the antibody can be directly labeled or detected with a labeled secondary antibody. That is, suitable, detectable labels for antibodies include direct labels, which label the antibody to the protein of interest, and indirect labels, which label an antibody that recognizes the antibody to the protein of interest.

These labels include radioisotopes, including, but not limited to ⁶⁴Cu, ⁶⁷Cu, ⁹⁰Y, ^{99m}Tc, ¹¹¹In, ¹²⁴I, ¹²⁵I, ¹³¹I, ¹³⁷Cs, ¹⁸⁶Re, ²¹¹At, ²¹²Bi, ²¹³Bi, ²²³Ra, ²⁴¹ Am, and ²⁴⁴Cm; enzymes having detectable products (for example, luciferase, peroxidase, alkaline phosphatase, β-galactosidase, and the like); fluorescers and fluorescent labels, for example, as provided herein; fluorescence emitting metals, for example, ¹⁵²Eu, or others of the lanthanide series, attached to the antibody through metal chelating groups such as EDTA; chemiluminescent compounds, for example, luminol, isoluminol, or acridinium salts; and bioluminescent compounds, for example, luciferin, or aequorin (green fluorescent protein), specific binding molecules, for example, magnetic particles, microspheres, nanospheres, and the like.

Alternatively, specific-binding pairs may be used, involving, for example, a second stage antibody or reagent that is detectably labeled and that can amplify the signal. For example, a primary antibody can be conjugated to biotin, and horseradish peroxidase-conjugated streptavidin added as a second stage reagent. Digoxin and antidigoxin provide another such pair. In other embodiments, the secondary antibody can be conjugated to an enzyme such as peroxidase in combination with a substrate that undergoes a color change in the presence of the peroxidase. The absence or presence of antibody binding can be determined by various methods, including flow cytometry of dissociated cells, microscopy, radiography, or scintillation counting. Such reagents and their methods of use are well known in the art.

Antibodies of the invention can be provided in the form of arrays, which are collections of plural biological molecules having locatable addresses that may be separately detectable. Generally, a microarray encompasses use of submicrogram quantities of biological molecules. The antibodies may be affixed to a substrate or may be in solution or suspension. The substrate can be porous or solid, planar or non-planar, unitary or distributed, such as a glass slide, a 96 well plate, with or without the use of microbeads or nanobeads. Antibody microarrays of the invention include arrays of LRRTM1 related antibodies obtained by purification, as fusion proteins, and or recombinantly, and can be used for specific binding studies (Zhu, H., et al. (2003) Curr. Opin. Chem. Biol. 7:55-63; Houseman, B. T., et al. (2002) Nature Biotechnol. 20:270-274; Schaeferling, M., et al. (2002) Electrophoresis 23:3097-3105; Weng, S., et al. (2002) Proteomics 2:48-57; Winssinger, N., et al. (2002) Proc. Natl. Acad. Sci. 99:11,139-11,144; Zhu, H., Bilgin, et al. (2001) Science 293:2101-2105; and MacBeath, G., et al. (2000) Science 289:1760-1763).

All of the immunogenic methods of the invention can be used alone or in combination with other conventional or unconventional therapies. For example, immunogenic molecules can be combined with other molecules that have a variety of antiproliferative effects, or with additional substances that help stimulate the immune response, for example, adjuvants or cytokines.

### Diagnostic and Therapeutic Applications

Diagnostic Applications

The invention is related to the use of LRRTM1 genes and gene products as part of a diagnostic assay for detecting diseases or susceptibility to diseases related to the presence of mutations in the nucleic acid sequences encoding a polypeptide of the present invention. Individuals carrying mutations in an LRRTM1 gene may be detected at the DNA level by a variety of techniques. Nucleic acids for diagnosis may be obtained from a patient sample. The genomic DNA may be used directly for detection or may be amplified enzymatically by using PCR, for example, as described by Saiki et al., Nature, 324: 163-166 (1986), prior to analysis. RNA or cDNA may also be used for the same purpose.

As an example, PCR primers complementary to the nucleic acid encoding a polypeptide of the present invention can be used to identify and analyze mutations. For example, deletions and insertions can be detected by a change in size of the amplified product in comparison to the normal genotype. Point mutations can be identified by hybridizing amplified DNA to radiolabeled RNA or alternatively, radiolabeled antisense DNA sequences. Perfectly matched sequences can be distinguished from mismatched duplexes by RNase A digestion or by differences in melting temperatures.

Genetic testing based on DNA sequence differences may be achieved by detecting alterations in electrophoretic mobility of DNA fragments in gels run with or without denaturing agents. Small sequence deletions and insertions can be visualized by high resolution gel electrophoresis. DNA fragments of different sequences may be distinguished on denaturing formamide gradient gels in which the mobilities of different DNA fragments are retarded in the gel at different positions according to their specific melting or partial melting temperatures, for example, as described by Myers et al., Science, 230:1242 (1985).

Sequence changes at specific locations may also be revealed by nuclease protection assays, such as RNase and SI protection or the chemical cleavage method as shown in Cotton et al., Proc. Natl. Acad. Sci., 85:4397-4401 (1985). Thus, the detection of a specific DNA sequence may be achieved by methods such as hybridization, RNase protection, chemical cleavage, direct DNA sequencing or the use of restriction enzymes, for example, restriction fragment length polymorphisms (RFLP) and Southern blotting of genomic DNA. In addition to more conventional gel-electrophoresis and DNA sequencing, mutations can also be detected by in situ analysis.

The invention also relates to a diagnostic assay for detecting altered levels of LRRTM1 proteins in various tissues. An over-expression of these proteins compared to normal control tissue samples may detect the presence of abnormal cellular proliferation, for example, a tumor. Assays used to detect protein levels in a host-derived sample are well-known to those of skill in the art and include radioimmunoassays, competitive-binding assays, Western Blot analysis, ELISA assays, “sandwich” assays, and other assays for the expression levels of the genes encoding the LRRTM1 proteins known in the art. Expression can be assayed by qualitatively or quantitatively measuring or estimating the level of LRRTM1 protein, or the level of mRNA encoding LRRTM1 protein, in a biological sample. Assays may be performed directly, for example, by determining or estimating absolute protein level or mRNA level, or relatively, by comparing the LRRTM1 protein or mRNA to a second biological sample. In performing these assays, the LRRTM1 protein or mRNA level in the first biological sample is measured or estimated and compared to a standard LRRTM1 protein level or mRNA level; suitable standards include second biological samples obtained from an individual not having the disorder of interest. Standards may be obtained by averaging levels of LRRTM1 in a population of individuals not having a disorder related to LRRTM1 expression. As will be appreciated in the art, once a standard LRRTM1 protein level or mRNA level is known, it can be used repeatedly as a standard for comparison.

Therapeutic Applications

The invention provides various therapeutic methods. In some embodiments, methods of modulating, including increasing and inhibiting, a biological activity of LRRTM1 are provided. In other embodiments, methods of modulating a signal transduction activity of LRRTM1 are provided. In further embodiments, methods of modulating interaction of LRRTM1 with another, interacting protein or other macromolecule (for example, DNA, carbohydrate, lipid), are provided.

Thus, in an embodiment, the therapeutic compositions herein are administered to subjects for treatment of a proliferative disease, such as a tumor for example, a tumor of the pancreas, ovary, colon, or rectum. In an embodiment, the therapeutic compositions herein are administered to subjects for modulation of immune related diseases. In a further embodiment, the therapeutic compositions herein are administered to subjects for modulation of apoptosis-related diseases.

As mentioned above, an effective amount of an agent of the invention is administered to the host. In an embodiment, the agent is administered at a dosage sufficient to produce a desired result. In some embodiments, the desired result is at least a reduction in a given biological activity of a subject polypeptide as compared to a control. In other embodiments, the desired result is an increase in the level of the active subject polypeptide (in the individual, or in a localized anatomical site in the individual), as compared to a control. In some embodiments, the desired result is at least a reduction in enzymatic activity of a subject polypeptide as compared to a control. In other embodiments, the desired result is an increase in the level of enzymatically active subject polypeptide (in the individual, or in a localized anatomical site in the individual), as compared to a control.

Typically, the compositions of the instant invention will contain from less than 1% to about 95% of the active ingredient, in some embodiments, about 10% to about 50%. Generally, between about 100 mg and 500 mg of the compositions will be administered to a child and between about 500 mg and 5 grams will be administered to an adult. Administration is generally by injection and often by injection to a localized area. The frequency of administration will be determined by the care given based on patient responsiveness. Other effective dosages can be readily determined by one of ordinary skill in the art through trials establishing dose response curves.

In order to calculate the amount of therapeutic agent to be administered, those skilled in the art could use readily available information with respect to the amount of agent necessary to have the desired effect. The amount of an agent necessary to increase or decrease a level of active LRRTM1 can be calculated from in vitro experimentation. The amount of agent will, of course, vary depending upon the particular agent used.

Other effective dosages can be readily determined by one of ordinary skill in the art through routine trials establishing dose response curves, for example, the amount of agent necessary to increase or decrease a level of active LRRTM1 can be calculated from in vitro experimentation. Those of skill will readily appreciate that dose levels can vary as a function of the specific compound, the severity of the symptoms, and the susceptibility of the subject to side effects, and preferred dosages for a given compound are readily determinable by those of skill in the art by a variety of means. For example, in order to calculate the polypeptide, polynucleotide, or modulator dose, those skilled in the art can use readily available information with respect to the amount necessary to have the desired effect, depending upon the particular agent used.

Proliferative Conditions

In some embodiments, LRRTM1 is involved in the control of cell proliferation, and an agent of the invention inhibits undesirable cell proliferation. Such agents are useful for treating disorders that involve abnormal cell proliferation, including, but not limited to, cancer. The polypeptides, polynucleotides, antibodies, and other agents of the invention are useful for treating, for example, ovarian cancer, pancreatic cancer, and/or colon/colorectal cancer, as described below in the Examples and FIGS. 2-5. Whether a particular agent and/or therapeutic regimen of the invention is effective in reducing unwanted cellular proliferation, for example, in the context of treating cancer, can be determined using standard methods.

The therapeutic compositions and methods of the invention can be used in the treatment of cancer and/or any abnormal malignant cell or tissue growth, for example, a tumor. In an embodiment, the compositions and methods of the invention kill tumor cells. In an embodiment, they inhibit tumor development. Cancer is characterized by the proliferation of abnormal cells that tend to invade the surrounding tissue and metastasize to new body sites. The growth of cancer cells exceeds that of and is uncoordinated with the normal cells and tissues. In an embodiment, the compositions and methods of the invention inhibit the progression of premalignant lesions to malignant tumors.

Cancer encompasses carcinomas, which are cancers of epithelial cells, and are the most common forms of human cancer; carcinomas include squamous cell carcinoma, adenocarcinoma, melanomas, and hepatomas. Cancer also encompasses sarcomas, which are tumors of mesenchymal origin, and includes osteogenic sarcomas, leukemias, and lymphomas. Cancers can have one or more than one neoplastic cell type. Some characteristics that can, in some instances, apply to cancer cells are that they are morphologically different from normal cells, and may appear anaplastic; they have a decreased sensitivity to contact inhibition, and may be less likely than normal cells to stop moving when surrounded by other cells; and they have lost their dependence on anchorage for cell growth, and may continue to divide in liquid or semisolid surroundings, whereas normal cells must be attached to a solid surface to grow.

Treatment herein refers to obtaining a desired pharmacologic and/or physiologic effect, covering any treatment of a pathological condition or disorder in a mammal, including a human. The effect may be prophylactic in terms of completely or partially preventing a disorder or symptom thereof and/or may be therapeutic in terms of a partial or complete cure for a disorder and/or adverse affect attributable to the disorder. Thus, the invention provides both treatment and prophylaxis. It includes preventing the disorder from occurring or recurring in a subject who may be predisposed to the disorder but is not yet symptomatic, inhibiting the disorder, such as arresting its development, stopping or terminating the disorder or at least symptoms associated therewith, so that the host no longer suffers from the disorder or its symptoms, such as causing regression of the disorder or its symptoms, for example, by restoring or repairing a lost, missing or defective function, or stimulating an inefficient process; or relieving, alleviating, or ameliorating the disorder, or symptoms associated therewith, where ameliorating is used in a broad sense to refer to at least a reduction in the magnitude of a parameter, such as inflammation, pain, and/or tumor size.

The polynucleotides, polypeptides, and antibodies described above can be used to treat cancer. In an embodiment, a fusion protein or conjugate can additionally comprise a tumor-targeting moiety. Suitable moieties include those that enhance delivery of an therapeutic molecule to a tumor. For example, compounds that selectively bind to cancer cells compared to normal cells, selectively bind to tumor vasculature, selectively bind to the tumor type undergoing treatment, or enhance penetration into a solid tumor are included in the invention. Tumor targeting moieties of the invention can be peptides. Nucleic acid and amino acid molecules of the invention can be used alone or as an adjunct to cancer treatment. For example, a nucleic acid or amino acid molecules of the invention may be added to a standard chemotherapy regimen. It may be combined with one or more of the wide variety of drugs that have been employed in cancer treatment, including, but are not limited to, cisplatin, taxol, etoposide, Novantrone (mitoxantrone), actinomycin D, camptohecin (or water soluble derivatives thereof), methotrexate, mitomycins (for example, mitomycin C), dacarbazine (DTIC), and anti-neoplastic antibiotics such as doxorubicin and daunomycin, or others, described, for example, in De Vota et a;/. 2-1. De Vita, V. T., Jr., et al., eds. (2001) Cancer: Principles & Practice of Oncology. Lippincott Williams & Wilkins.

Drugs employed in cancer therapy may have a cytotoxic or cytostatic effect on cancer cells, or may reduce proliferation of the malignant cells. Drugs employed in cancer treatment can also be peptides. A nucleic acid or amino acid molecules of the invention can be combined with radiation therapy. A nucleic acid or amino acid molecule of the invention may be used adjunctively with therapeutic approaches described in De Vita et al., 2001. For those combinations in which a nucleic acid or amino acid molecule of the invention and a second anti-cancer agent exert a synergistic effect against cancer cells, the dosage of the second agent may be reduced, compared to the standard dosage of the second agent when administered alone. A method for increasing the sensitivity of cancer cells comprises co-administering a nucleic acid or amino acid molecule of the invention with an amount of a chemotherapeutic anti-cancer drug that is effective in enhancing sensitivity of cancer cells. Co-administration may be simultaneous or non-simultaneous administration. A nucleic acid or amino acid molecule of the invention may be administered along with other therapeutic agents, during the course of a treatment regimen. In one embodiment, administration of a nucleic acid or amino acid molecule of the invention and other therapeutic agents is sequential. An appropriate time course may be chosen by the physician, according to such factors as the nature of a patient's illness, and the patient's condition.

The invention also provides a method for prophylactic or therapeutic treatment of a subject needing or desiring such treatment by providing a vaccine that can be administered to the subject. The vaccine may comprise one or more agent of the invention, for example an antibody vaccine composition, a polypeptide vaccine composition, or a polynucleotide vaccine composition, useful for preventing or treating proliferative disorders, obesity, cardiac hypertrophy, or liver disease.

In some embodiments, LRRTM1 is involved in the control of cell proliferation, and an agent of the invention inhibits undesirable cell proliferation. Such agents are useful for treating disorders that involve abnormal cell proliferation, including, but not limited to, lung, colorectal, breast, bladder, pancreatic, and stomach cancer. Whether a particular agent and/or therapeutic regimen of the invention is effective in reducing unwanted cellular proliferation, for example, in the context of treating cancer, can be determined using standard methods. For example, the number of cancer cells in a biological sample (for example, blood, a biopsy sample, and the like), can be determined. The tumor mass can be determined using standard radiological or biochemical methods.

Modulators of LRRTM1 find use in immunotherapy of neoplastic, paraneoplastic, and hyperproliferative disorders, including cancer and psoriasis. That is, the subject molecules can correspond to tumor antigens, of which at least 1770 have been identified (Yu and Restifo (2002) J. Clin. Inves. 110-289-294). Immunotherapeutic approaches include passive immunotherapy and vaccine therapy and can accomplish both generic and antigen-specific cancer immunotherapy.

Passive immunity approaches involve antibodies of the invention that are directed toward specific tumor-associated antigens. Such antibodies can eradicate systemic tumors at multiple sites, without eradicating normal cells. In some embodiments, the antibodies are combined with cytotoxic components, such as radioactive or chemotherapeutic components, as provided above, for example, combining the antibody's ability to specifically target tumors with the added lethality of the radioisotope to the tumor DNA.

Useful antibodies bind to or react with antigens comprising one or more discrete epitope or a combination of nested epitopes, for example, a 10-mer epitope and associated peptide multimers incorporating all potential 8-mers and 9-mers, or overlapping epitopes (Dutoit et al., J. Clin. Invest. 110:1813-1822 (2002)). Thus a single antibody can interact with one or more epitopes. Further, the antibody can be used alone or in combination with different antibodies that recognize either a single or multiple epitopes.

Neutralizing antibodies, described above, can provide therapy for cancer and proliferative disorders. Neutralizing antibodies that specifically recognize a protein or peptide of the invention can bind to the protein or peptide, for example, in a bodily fluid or the extracellular space, thereby modulating the biological activity of the protein or peptide. For example, neutralizing antibodies specific for proteins or peptides that play a role in stimulating the growth of cancer cells can be useful in modulating the growth of cancer cells. Similarly, neutralizing antibodies specific for proteins or peptides that play a role in the differentiation of cancer cells can be useful in modulating the differentiation of cancer cells.

Apoptosis and Cell Death

The control of cell numbers in mammals is believed to be determined, in part, by a balance between cell proliferation and cell death. One form of cell death, sometimes referred to as necrotic cell death, is typically characterized as pathologic, resulting from trauma or injury. In contrast, there is another, physiologic, form of cell death that usually proceeds in an orderly or controlled manner. This orderly or controlled form of cell death is often referred to as apoptosis (Barr, P. J., et al. (1994) Bio/Technology 12:487-493; Steller, H. (1995) Science, 267:1445-1449).

Apoptotic cell death naturally occurs in many physiological processes, including embryonic development and clonal selection in the immune system (Itoh, N., et al. (1991) Cell 66:233-243). Decreased levels of apoptotic cell death have been associated with a variety of pathological conditions, including cancer and immune disease (Thompson, C. B. (1995) Science 267:1456-1462). Antibodies specific to LRRTM1 can induce the apoptotic-induced death of cancer cells by binding to the extracellular domain.

Apoptosis can be assayed using any known method. Assays can be conducted on cell populations or an individual cell, and include morphological assays and biochemical assays. Procedures to detect cell death based on the TUNEL method are available commercially, for example, from Boehringer Mannheim (Cell Death Kit) and Oncor (Apoptag Plus).

Vaccine Therapy

The LRRTM1 polypeptide, including the extracellular domain of the mature form of LRRTM1, or portions of it, can be formulated and administered as a vaccine. Such a vaccine can be used as to treat patients overexpressing LRRTM1 at the surface of cancer cells, inducing antibody or cell mediated immune responses against the cancer cells, including antibody-dependent cell cytotoxicity (ADCC) or complement-dependent cytotoxicity (CDC).

The invention also provides a method for prophylactic or therapeutic treatment of a subject needing or desiring such treatment by providing a vaccine and administering the vaccine to the subject. The vaccine may comprise one or more of a polynucleotide, polypeptide, or modulator of the invention, for example an antibody vaccine composition, a polypeptide vaccine composition, or a polynucleotide vaccine composition. It may comprise a complement, biologically active fragment, or variant of any of these. For example, the vaccine can be a cancer vaccine, and the polypeptide can concomitantly be a cancer antigen. The vaccine can be administered with or without an adjuvant.

Vaccine therapy involves the use of polynucleotides, polypeptides, or agents of the invention as immunogens for tumor antigens (Machiels et al., 2002; Shinnick, T. M., et al. (1983) Ann. Rev. Microbiol. 37:425-446). For example, peptide-based vaccines of the invention include unmodified subject polypeptides, fragments thereof, and MHC class I and class II-restricted peptide (Knutson et al., 2001), comprising, for example, the disclosed sequences with universal, nonspecific MHC class II-restricted epitopes. Peptide-based vaccines comprising a tumor antigen can be given directly, either alone or in conjunction with other molecules. The vaccines can also be delivered orally by producing the antigens in transgenic plants that can be subsequently ingested (U.S. Pat. No. 6,395,964).

In some embodiments, antibodies themselves can be used as antigens in anti-idiotype vaccines. That is, administering an antibody to a tumor antigen can stimulate B cells to make antibodies to that antibody, which in turn recognize the tumor cells.

Nucleic acid-based vaccines can deliver tumor antigens as polynucleotide constructs encoding the antigen. Vaccines comprising genetic material, such as DNA or RNA, can be given directly, either alone or in conjunction with other molecules. Administration of a vaccine expressing a molecule of the invention, for example, as plasmid DNA, leads to persistent expression and release of the therapeutic immunogen over a period of time, helping to control unwanted tumor growth.

In some embodiments, nucleic acid-based vaccines encode subject antibodies. In such embodiments, the vaccines (for example, DNA vaccines) can include post-transcriptional regulatory elements, such as the post-transcriptional regulatory acting RNA element (WPRE) derived from Woodchuck Hepatitis Virus. These post-transcriptional regulatory elements can be used to target the antibody, or a fusion protein comprising the antibody and a co-stimulatory molecule, to the tumor microenvironment (Pertl et al., Blood 101:649 (2003)).

Besides stimulating anti-tumor immune responses by inducing humoral responses, vaccines of the invention can also induce cellular responses, including stimulating T-cells that recognize and kill tumor cells directly. For example, nucleotide-based vaccines of the invention encoding tumor antigens can be used to activate the CD8⁺ cytotoxic T lymphocyte arm of the immune system.

In some embodiments, the vaccines activate T-cells directly, and in others they enlist antigen-presenting cells to activate T-cells. Killer T-cells are primed, in part, by interacting with antigen-presenting cells, for example, dendritic cells. In some embodiments, plasmids comprising the nucleic acid molecules of the invention enter antigen-presenting cells, which in turn display the encoded tumor-antigens that contribute to killer T-cell activation. Again, the tumor antigens can be delivered as plasmid DNA constructs, either alone or with other molecules.

In further embodiments, RNA can be used. For example, antigen-presenting cells can be transfected or transduced with RNA encoding tumor antigens (Heiser et al., J. Clin. Invest. 109:409-417 (2002); Mitchell et al., J. Clin. Invest. 106:1065-1069 (2000). This approach overcomes the limitations of obtaining sufficient quantities of tumor material, extending therapy to patients otherwise excluded from clinical trials. For example, a subject RNA molecule isolated from tumors can be amplified using RT-PCR. In some embodiments, the RNA molecule of the invention is directly isolated from tumors and transfected into antigen-presenting cells or dendritic cells with no intervening cloning steps.

In some embodiments, the molecules of the invention are altered such that the peptide antigens are more highly antigenic than in their native state. These embodiments address the need in the art to overcome the poor in vivo immunogenicity of most tumor antigens by enhancing tumor antigen immunogenicity via modification of epitope sequences (Yu and Restifo, 2002).

Another recognized problem of cancer vaccines is the presence of preexisting neutralizing antibodies. Some embodiments of the present invention overcome this problem by using viral vectors from non-mammalian natural hosts, i.e., avian pox viruses. Alternative embodiments that also circumvent preexisting neutralizing antibodies include genetically engineered influenza viruses, and the use of “naked” plasmid DNA vaccines that contain DNA with no associated protein. (Yu and Restifo, 2002).

### Therapeutic Compositions and Formulations

Routes of Administration and Carriers

The LRRTM1 inhibitors of the invention can be administered in vivo by a variety of routes, including intravenous, intra-arterial, subcutaneous, parenteral, intranasal, intramuscular, intracardiac, intraventricular, intratracheal, buccal, rectal, intraperitoneal, intradermal, topical, transdermal, and intrathecal, or otherwise by implantation or inhalation. They may be administered in formulations, as described in more detail below. They may be administered in powder form intranasally or by inhalation. They may be administered as suppositories, for example, as formulated by mixing with a variety of bases, such as emulsifying bases, water-soluble bases, cocoa butter, carbowaxes, and polyethylene glycols; which melt at body temperature, yet are solidified at room temperature. Jet injection can be used for intramuscular or intradermal administration (Furth et al., Anal. Biochem. 205:365-368 (1992)). The DNA can be coated onto gold microparticles and delivered intradermally by a particle bombardment device, or “gene gun” as described in the literature (Tang et al., Nature 356:152-154 (1992)), where gold microprojectiles are coated with the DNA, then bombarded into skin cells. These methods of in vivo administration are known in the art.

In some embodiments, therapeutic compositions are provided in formulation with pharmaceutically acceptable carriers, a wide variety of which are known in the art (Gennaro, Remington: The Science and Practice of Pharmacy with Facts and Comparisons: Drug facts Plus, 20th ed. (2003); Ansel et al., Pharmaceutical Dosage Forms and Drug Delivery Systems, 7^{th} ed., Lippencott Williams and Wilkins (2004); Kibbe et al., Handbook of Pharmaceutical Excipients, 3^{rd} ed., Pharmaceutical Press (2000)). Pharmaceutically acceptable carriers, such as vehicles, adjuvants, carriers, or diluents, are available to the public. Moreover, pharmaceutically acceptable auxiliary substances, such as pH adjusting and buffering agents, tonicity adjusting agents, stabilizers, wetting agents and the like, are available to the public.

The inhibitors of the invention may be employed in combination with a suitable pharmaceutical carrier to comprise a pharmaceutical composition for parenteral administration. Accordingly, the invention provides a composition comprising an LRRTM1 inhibitor of the invention and a pharmaceutically acceptable carrier. Such compositions comprise a therapeutically effective amount of the inhibitor, and a pharmaceutically acceptable carrier. Such a carrier includes, but is not limited to, saline, buffered saline, dextrose, water, glycerol, ethanol, and combinations thereof. The formulation should suit the mode of administration.

In pharmaceutical dosage, the therapeutic compositions can be administered in the form of their pharmaceutically acceptable salts, either alone or in appropriate association or combination with other pharmaceutically active compounds. The therapeutic compositions are formulated in accordance with the mode of administration. Thus, the subject compositions can be formulated into preparations in solid, semi-solid, liquid, or gaseous forms, such as tablets, capsules, powders, granules, ointments, solutions, suppositories, enemas, injections, inhalants, and aerosols. The methods and excipients cited herein are merely exemplary and are in no way limiting.

The agents, polynucleotides, and polypeptides can be formulated into preparations for injection by dissolving, suspending, or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives. They may be formulated into preparations for administration via inhalation, for example as formulated into pressurized acceptable propellants such as dichlorodifluoromethane, propane, nitrogen, and the like. The therapeutic compositions of the invention can be formulated into a sustained release microcapsules, such as with biodegradable or non-biodegradable polymers, using techniques known in the art. An example of a biodegradable formulation suitable for use herein includes poly lactic acid-glycolic acid polymer. An example of a non-biodegradable formulation suitable for use herein includes a polyglycerin fatty acid ester. A method of making these formulations is described in, for example, EP 1 125 584 A1. Other formulations for parenteral delivery can also be used, as conventional in the art.

The therapeutic compositions of the invention will be formulated and dosed in a fashion consistent with good medical practice, taking into account the clinical condition of the individual subject, the site of delivery of the fusion molecule composition, the method of administration, the scheduling of administration, and other factors known to practitioners. The effective amount of FGFR fusion molecule for purposes herein is thus determined by such considerations.

Unit dosage forms can be provided wherein each dosage unit contains a predetermined amount of the composition containing one or more agents. In an embodiment, a therapeutic composition is supplied in single-use prefilled syringes for injection. The composition may comprise saline, sucrose, or the like; a buffer, such as phosphate, or the like; and be formulated within a stable and effective pH range. In an embodiment, a therapeutic composition is provided as a lyophilized powder in a multiple-use vial, which can be reconstituted upon addition of an appropriate liquid, for example, sterile bacteriostatic water. In an embodiment, a therapeutic composition comprises one or more substances that inhibit protein aggregation, including, but not limited to, sucrose or arginine. In an embodiment, a composition of the invention comprises heparin and/or a proteoglycan.

These pharmaceutical compositions are administered in an amount effective for treatment and/or prophylaxis of the specific indication. The effective amount is typically dependent on the weight of the subject being treated, his or her physical or health condition, the extensiveness of the condition to be treated, and/or the age of the subject being treated. In general, the protein therapeutics of the invention are to be administered in an amount in the range of about 5 ug/kg body weight to about 10 mg/kg body weight per dose. Optionally, the protein therapeutics of the invention can be administered in an amount in the range of about 10 ug/kg body weight to about 9 mg/kg body weight per dose. Further optionally, the protein therapeutics of the invention can be administered in an amount in the range of about 100 ug/kg body weight to about 8 mg/kg body weight per dose. Still optionally, the FGFR fusion proteins of the invention can be administered in an amount in the range of about 1 mg/kg body weight to about 7 mg/kg body weight per dose.

The therapeutic compositions of the invention can be administered as needed to subjects in need of cancer therapy or prophylaxis. Determination of the frequency of administration can be made by persons skilled in the art, such as an attending physician based on considerations of the condition being treated, age of the subject being treated, severity of the condition being treated, general state of health of the subject being treated and the like. In one embodiment, an effective dose of the therapeutic is administered to a subject one or more times.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims. Moreover, advantages described in the body of the specification, if not included in the claims, are not per se limitations to the claimed invention.

It is to be understood that both the foregoing general description and the following detailed description are exemplary and explanatory only and are not restrictive of the invention, as claimed. Moreover, it must be understood that the invention is not limited to the particular embodiments described, as such may, of course, vary. Further, the terminology used to describe particular embodiments is not intended to be limiting, since the scope of the present invention will be limited only by its claims. The claims do not encompass embodiments in the public domain.

With respect to ranges of values, the invention encompasses each intervening value between the upper and lower limits of the range to at least a tenth of the lower limit's unit, unless the context clearly indicates otherwise. Further, the invention encompasses any other stated intervening values. Moreover, the invention also encompasses ranges excluding either or both of the upper and lower limits of the range, unless specifically excluded from the stated range.

Unless defined otherwise, the meanings of all technical and scientific terms used herein are those commonly understood by one of ordinary skill in the art to which this invention belongs. One of ordinary skill in the art will also appreciate that any methods and materials similar or equivalent to those described herein can also be used to practice or test the invention. Further, all publications mentioned herein are incorporated by reference in their entireties.

The specification is most thoroughly understood in light of the following references, all of which are hereby incorporated in their entireties. The disclosures of the patents and other references cited above are also hereby incorporated by reference. The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### EXAMPLES

The examples, which are intended to be purely exemplary of the invention and should therefore not be considered to limit the invention in any way, also describe and detail aspects and embodiments of the invention discussed above. The examples are not intended to represent that the experiments below are all or the only experiments performed. Efforts have been made to ensure accuracy with respect to numbers used (for example, amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### LRRTM1 Gene Expression Analysis of Normal and Cancerous Tissues Using Microarrays

The differential level of gene expression was compared in individual human cancer tissue specimens by screening a proprietary Five Prime Therapeutics, Inc. microarray chip and an Affymetrix microarray chip and interrogating a proprietary oncology database from GeneLogic. The Affymetrix GeneChip® array platform, the Human Genome U133 and U133Plus_{—}2 (Affymetrix, Inc, Santa Clara, Calif.) was interrogated with probe 238815_at.

RNA was prepared from tumor tissue resected from eight patients with ovarian cancer and from normal-appearing adjacent tissue resected from three of the same patients. RNA was also prepared from 35 other normal tissue specimens. Tissues were flash frozen in liquid nitrogen, transported on dry ice, and stored at minus 180° C. in liquid nitrogen. Histology was performed on a sample of each frozen tissue specimen and reviewed by a pathologist to confirm the cancer diagnosis or the tissue's normality. Only confirmed specimens were used for microarray hybridization or real time PCR experiments.

RNA was isolated from the tissues by grinding them to a fine powder under liquid nitrogen with a pre-chilled mortar and pestle. Total RNA was extracted using TRIzol reagent (Invitrogen, Carlsbad, Calif., USA) according to the manufacturer's protocol. It was treated with DNase in a final volume of 500 μl using 350 μg total RNA, 35 U DNase I, 50 μL DNase buffer and 280U RNaseOUT (all from Invitrogen). Following incubation at 37° C. for 30 min., 500 μl phenol:chloroform:isoamyl alcohol (Invitrogen) was added, and the mixture vortexed, centrifuged at 14,000 rpm for 5 min., and the aqueous phase transferred to a new 2 ml tube. The RNA was then ethanol precipitated by adding 80 μL 5 M NH₄OAc, 1.5 ml EtOH, incubated at −20° C. for 30 min., then centrifuged at 14,000 rpm for 30 min. The pellet was washed with 75% EtOH and resuspended with 20 μL H₂0. The quality and concentration of the RNA were determined spectrophotometrically at 260 and 280 nm wavelengths and by agarose gel electrophoresis.

The resulting RNA was used as a template to prepare cDNA. cDNA synthesis was performed in a final volume of 100 μl with 2 μg total RNA, random hexamer primers and oligo(dT)₁₆ primer at a final concentration of 2 mM each, 10 μl of reverse transcriptase buffer, 22 μl of 25 mM MgCl₂, 20 μl of 10 mM dNTP mix, 40 U RNase inhibitor and 125 U Multiscribe reverse transcriptase (all from Applied Biosystems, Foster City, Calif., USA). This mixture was incubated at 25° C. for 10 min., at 42° C. for 60 min., and then at 95° C. for 5 min.

### Example 2

### Expression of LRRTM1 Quantified by Real-Time PCR

RNA was prepared from normal and cancerous tissues and a subset of these tissues were used to perform real-time PCR. Complementary DNA was prepared as described in Example 1. PCR primers and probes were designed using Primer Express™ software (Applied Biosystems, Foster City, Calif., USA). The sequences used for designing PCR primers and probes were limited to exon 2 of LRRTM1 (NCBI Protein IDs: 28175111 and 16552104). The sequences for the primers and probe are: primer forward GTCACGCAGCGCAGGAA (SEQ ID NO: 29); primer reverse GACATGGCAGCCATCTGATG (SEQ ID NO: 30); and probe: 6FAM-AAAGCAGAAACAGACCAT (SEQ ID NO: 31).

Samples were run in duplicate in a 25-μL reaction volume containing 2× TaqMan Universal PCR Master Mix (Applied Biosystems), primers at a final concentration of 900 nM each, 250 nM probe, water to a 20-μL final volume, and 5-μL of the cDNA. The PCR was run in the ABI Prism 7000 Sequence Detection System (Applied Biosystems) using the following amplification parameters: 2 min at 50° C., 10 min at 95° C., 40 cycles of 15 s at 95° C. and 1 min at 60° C.

To confirm that the RT-PCR primer-probes were specific for LRRTM1 ORF, the probes and primers were tested on a cDNA plasmid clone that encoded LRRTM1. As shown in FIG. 8, when RT-PCR primer-probes for LRRTM1 ORF reacted with cDNA plasmid clone CLN00369332, which encodes LRRTM1, the resulting PCR signal was robust.

The real-time PCR data shown in FIG. 2 demonstrates that three of the eight ovarian cancer samples exhibited higher LRRTM1 expression compared to normal ovarian tissue taken from sites adjacent to the tumors. In addition, FIG. 3 shows that LRRTM1 expression in the several normal, adult, non-pregnant tissue types tested is low to undetectable.

### Sequence Listing

The instant application contains a Sequence Listing which has been submitted via 1 diskette and a printed paper copy, and is hereby incorporated by reference in its entirety. Said diskette, recorded on Oct. 24, 2006, contains file 89463304.txt.

The Sequence Listing provides polynucleotide sequences that encode polypeptides of the invention (SEQ. ID. NO. (N1)). SEQ. ID. NO.:27 represents the complete nucleotide sequence of the LRRTM1, including both coding and non-coding regions, which is identified as NP_{—}849161.1:NM_{—}178839.3 in the NCBI database. SEQ. ID. NO.:13 represents the nucleotide sequences of the open reading frame of LRRTM1, which encodes the polypeptides of the invention identified herein as clone ID No. 16552104:16552103.

The Sequence Listing also provides amino acid sequences of polypeptides of the invention (SEQ. ID. NO. (P1)). SEQ. ID. NO. 28 represents the amino acid sequence of the LRRTM1 which is identified as NP_{—}849161.1:NM_{—}178839.3 in the NCBI database. SEQ. ID. NOS.:14-25 represent the amino acid sequences of fragments of LRRTM1, used as described herein. SEQ. ID. NO. 26 represents the amino acid sequence of the LRRTM1 which is identified herein as clone ID No. 16552104:16552103.

TABLE 1
SEQ ID NOS.: 1-28

| | SEQ. ID. | SEQ. ID. | | |
|---|---|---|---|---|
| FP ID | NO. (N1) | NO. (P1) | Clone ID | Sequence Fragment |
| HG1019738 | SEQ. ID. | SEQ. ID. | 16552104_aa62-85_lrr1 | HNLSGLLGLSLRYNSLSEL |
| | NO. 1 | NO. 14 | | RAGQF |
| | | | | (SEQ ID NO: 14) |
| HG1019739 | SEQ. ID. | SEQ. ID. | 16552104_aa86-109_lrr2 | TGLMQLTWLYLDHNHICS |
| | NO. 2 | NO. 15 | | VQGDAF |
| | | | | (SEQ ID NO: 15) |
| HG1019740 | SEQ. ID. | SEQ. ID. | 16552104_aa110-133_lrr3 | QKLRRVKELTLSSNQITQL |
| | NO. 3 | NO. 16 | | PNTTF |
| | | | | (SEQ ID NO: 16) |
| HG1019741 | SEQ. ID. | SEQ. ID. | 16552104_aa134-157_lrr4 | RPMPNLRSVDLSYNKLQA |
| | NO. 4 | NO. 17 | | LAPDLF |
| | | | | (SEQ ID NO: 17) |
| HG1019742 | SEQ. ID. | SEQ. ID. | 16552104_aa158-181_lrr5 | HGLRKLTTLHMRANAIQF |
| | NO. 5 | NO. 18 | | VPVRIF |
| | | | | (SEQ ID NO: 18) |
| HG1019743 | SEQ. ID. | SEQ. ID. | 16552104_aa182-205_lrr6 | QDCRSLKFLDIGYNQLKSL |
| | NO. 6 | NO. 19 | | ARNSF |
| | | | | (SEQ ID NO: 19) |
| HG1019744 | SEQ. ID. | SEQ. ID. | 16552104_aa206-229_lrr7 | AGLFKLTELHLEHNDLVK |
| | NO. 7 | NO. 20 | | VNFAHF |
| | | | | (SEQ ID NO: 20) |
| HG1019745 | SEQ. ID. | SEQ. ID. | 16552104_aa230-253_lrr8 | PRLISLHSLCLRRNKVAIV |
| | NO. 8 | NO. 21 | | VSSLD |
| | | | | (SEQ ID NO: 21) |
| HG1019746 | SEQ. ID. | SEQ. ID. | 16552104_aa253-276_lrr9 | DWVWNLEKMDLSGNEIE |
| | NO. 9 | NO. 22 | | YMEPHVF |
| | | | | (SEQ ID NO: 22) |
| HG1019747 | SEQ. ID. | SEQ. ID. | 16552104_aa277-300_lrr10 | ETVPHLQSLQLDSNRLTYI |
| | NO. 10 | NO. 23 | | EPRIL |
| | | | | (SEQ ID NO: 23) |
| HG1019748 | SEQ. ID. | SEQ. ID. | 16552104_aa34-58_n_term | AAPSGCPQLCRCEGRLLY |
| | NO. 11 | NO. 24 | frag | CEALNET |
| | | | | (SEQ ID NO: 24) |
| HG1019749 | SEQ. ID. | SEQ. ID. | 16552104_aa306-346_c_term | LTSITLAGNLWDCGRNVC |
| | NO. 12 | NO. 25 | frag | ALASWLNNFQGRYDGNL |
| | | | | QCASPE |
| | | | | (SEQ ID NO: 25) |
| HG1019750 | SEQ. ID. | SEQ. ID. | 16552104:16552103 | |
| | NO. 13 | NO. 26 | | |
| | SEQ. ID. | SEQ. ID. | NP_849161.1:NM_178839.3 | |
| | NO. 27 | NO. 28 | | |

TABLE 2
Post-Translational Modifications

| | | Post-translational |
|---|---|---|
| Position | Sequence | modification |
| 56-59 | NLTE | N-glycosylation site |
| | (SEQ ID NO: 32) | |
| 63-66 | NLSG | N-glycosylation site |
| | (SEQ ID NO: 33) | |
| 130-133 | NTTF | N-glycosylation site |
| | (SEQ ID NO: 34) | |
| 380-383 | NRSD | N-glycosylation site |
| | (SEQ ID NO: 35) | |
| 263-277 | lsgneieYmephvfe | Tyrosine sulfation site |
| | (SEQ ID NO: 36) | |
| 340-354 | lqcaspeYaggedvl | Tyrosine sulfation site |
| | (SEQ ID NO: 37) | |
| 15-18 | RRpS | cAMP- and cGMP-dependent |
| | (SEQ ID NO: 38) | protein kinase phosphory- |
| | | lation site |
| 161-164 | RKlT | cAMP- and cGMP-dependent |
| | (SEQ ID NO: 39) | protein kinase phosphory- |
| | | lation site |
| 71-73 | SlR | Protein kinase C |
| | | phosphorylation site |
| 132-134 | TfR | Protein kinase C |
| | | phosphorylation site |
| 186-188 | SlK | Protein kinase C |
| | | phosphorylation site |
| 289-291 | SnR | Protein kinase C |
| | | phosphorylation site |
| 302-304 | SwK | Protein kinase C |
| | | phosphorylation site |
| 379-381 | TnR | Protein kinase C |
| | | phosphorylation site |
| 449-451 | SwK | Protein kinase C |
| | | phosphorylation site |
| 456-458 | SlR | Protein kinase C |
| | | phosphorylation site |
| 466-468 | TqR | Protein kinase C |
| | | phosphorylation site |
| 76-79 | SlsE | Casein kinase II |
| | (SEQ ID NO: 40) | phosphorylation site |
| 250-253 | SslD | Casein kinase II |
| | (SEQ ID NO: 41) | phosphorylation site |
| 264-267 | SgnE | Casein kinase II |
| | (SEQ ID NO: 42) | phosphorylation site |
| 293-296 | TyiE | Casein kinase II |
| | (SEQ ID NO: 43) | phosphorylation site |
| 393-396 | TlaD | Casein kinase II |
| | (SEQ ID NO: 44) | phosphorylation site |
| 484-487 | SaqE | Casein kinase II |
| | (SEQ ID NO: 45) | phosphorylation site |
| 503-506 | TinE | Casein kinase II |
| | (SEQ ID NO: 46) | phosphorylation site |
| 140-146 | Rsv.Dls.Y | Tyrosine kinase |
| | (SEQ ID NO: 47) | phosphorylation site |
| 188-194 | Kfl.Dig.Y | Tyrosine kinase |
| | (SEQ ID NO: 48) | phosphorylation site |
| 19-24 | GVvlCL | N-myristoylation site |
| | (SEQ ID NO: 49) | |
| 83-88 | GQftGL | N-myristoylation site |
| | (SEQ ID NO: 50) | |
| 338-343 | GNlqCA | N-myristoylation site |
| | (SEQ ID NO: 51) | |
| 366-371 | GAepTS | N-myristoylation site |
| | (SEQ ID NO: 52) | |
| 400-405 | GQhdGT | N-myristoylation site |
| | (SEQ ID NO: 53) | |
| 415-420 | GGehAE | N-myristoylation site |
| | (SEQ ID NO: 54) | |
| 499-504 | GAlvTI | N-myristoylation site |
| | (SEQ ID NO: 55) | |
| 508-513 | GSctCH | N-myristoylation site |
| | (SEQ ID NO: 56) | |

TABLE 3
Clone ID 16552104 Characteristics

| | |
|---|---|
| FP ID | HG101975 |
| Clone ID | 16552104 |
| Cluster | 185918 |
| Classification | single transmembrane |
| Predicted Protein Length | 522 |
| Treevote | 0.03 |
| Signal Peptide Coordinates | 5-19 |
| Mature Peptide Coordinates | 20-522 |
| Alternative Signal Peptide Coordinates | 18-30; 22-34 |
| Alternative Mature Peptide | 31-522; 35-522 |
| No. of Transmembrane Domains | 1 |
| Transmembrane Coordinates | 428-450 |
| Non-transmembrane Coordinates | 1-427; 451-522 |
| Pfam Domains | LRR |

TABLE 4
Predicted Functional Domain Coordinates

| Domain | Coordinates |
|---|---|
| LRR1 beta sheet and beta/alpha turn | 62-85 |
| LRR2 beta sheet and beta/alpha turn | 86-109 |
| LRR3 beta sheet and beta/alpha turn | 110-133 |
| LRR4 beta sheet and beta/alpha turn | 134-157 |
| LRR5 beta sheet and beta/alpha turn | 158-181 |
| LRR6 beta sheet and beta/alpha turn | 182-205 |
| LRR7 beta sheet and beta/alpha turn | 206-229 |
| LRR8 beta sheet and beta/alpha turn | 230-253 |
| LRR9 beta sheet and beta/alpha turn | 253-276 |
| LRR10 beta sheet and beta/alpha turn | 277-300 |
| N-terminal beta finger | 34-58 |
| C-terminal beta loop | 306-346 |

1. A method of diagnosing cancer in a subject, or assigning a prognostic risk of one or more future clinical outcomes to a subject suffering from cancer, comprising:
performing an assay configured to detect a soluble form of LRRTM1 in a body fluid sample obtained from the subject;
obtaining a result from the assay; and
relating the result of the assay to the presence or absence of cancer in the subject, or to the prognostic risk of one or more clinical outcomes for the subject.

2. The method of claim 1, wherein the soluble form of LRRTM1 comprises all or a portion of the extracellular domain of full length LRRTM1, the full length LRRTM1 having the sequence depicted in SEQ. ID. NO.:26.

3. The method of claim 1, wherein the cancer is ovarian cancer.

4. The method of claim 1, wherein the cancer is pancreatic cancer.

5. The method of claim 1, wherein the cancer is colon cancer.

6. The method of claim 2, wherein performing the assay comprises contacting the body fluid sample with an antibody that binds to the extracellular domain of full length LRRTM1, and detecting polypeptide binding to the antibody.

7. The method of claim 6, wherein the assay is a sandwich immunoassay.

8. The method of claim 1, wherein the body fluid sample is obtained from blood, serum, or plasma.

9. The method of claim 1, wherein the assay result is expressed as an amount of the soluble form of LRRTM1, and the relating step comprises comparing the assay result to a predetermined threshold level of the soluble form of LRRTM1, and performing one or more of the following determinations:
diagnosing the presence of cancer in the subject if the assay result is greater than the threshold level; or
diagnosing the absence of cancer in the subject if the assay result is less than the threshold level; or
assigning an increased likelihood of a poor prognostic outcome if the assay result is greater than the threshold level, relative to the prognostic risk assigned; or
assigning a decreased likelihood of a poor prognostic outcome if the assay result is less than the threshold level relative to the prognostic risk assigned.

10. A method of determining the presence of a polypeptide specifically binding to an antibody in a sample, comprising:
allowing an antibody to interact with the sample; and
determining whether interaction between the antibody and the polypeptide has occurred;
wherein the antibody specifically recognizes, binds to, interferes with, and/or modulates the biological activity of at least one polypeptide, polynucleotide and/or as shown in the Tables, Sequence Listing, or Figures, wherein the polypeptide is other than a full-length LRRTM1 of SEQ. ID. NO.:26.

11. A method of determining the presence of an antibody specifically binding to a polypeptide or a polynucleotide in a sample, comprising:
allowing an isolated polynucleotide encoding a polypeptide or an isolated polypeptide, wherein the polypeptide comprises an amino acid sequence or one or more biologically active fragments thereof, as shown in the Tables, Figures, or Sequence Listing, to interact with the sample; and
determining whether interaction between the antibody and the polypeptide or polynucleotide has occurred.

12.-80. (canceled)


SEQUENCE LISTING (57 sequences)

SEQ ID NO: 1 - DNA, 72 bp, Homo sapiens
cacaacctgtccggcctgctgggcttgtccctgcgctacaacagcctctcggagctgcgcgccggccagttc

SEQ ID NO: 2 - DNA, 72 bp, Homo sapiens
acggggttaatgcagctcacgtggctctatctggatcacaatcacatctgctccgtgcagggggacgccttt

SEQ ID NO: 3 - DNA, 72 bp, Homo sapiens
cagaaactgcgccgagttaaggaactcacgctgagttccaaccagatcacccaactgcccaacaccaccttc

SEQ ID NO: 4 - DNA, 72 bp, Homo sapiens
cggcccatgcccaacctgcgcagcgtggacctctcgtacaacaagctgcaggcgctcgcgcccgacctcttc

SEQ ID NO: 5 - DNA, 72 bp, Homo sapiens
cacgggctgcggaagctcaccacgctgcatatgcgggccaacgccatccagtttgtgcccgtgcgcatcttc

SEQ ID NO: 6 - DNA, 72 bp, Homo sapiens
caggactgccgcagcctcaagtttctcgacatcggatacaatcagctcaagagtctggcgcgcaactctttc

SEQ ID NO: 7 - DNA, 72 bp, Homo sapiens
gccggcttgtttaagctcaccgagctgcacctcgagcacaacgacttggtcaaggtgaacttcgcccacttc

SEQ ID NO: 8 - DNA, 72 bp, Homo sapiens
ccgcgcctcatctccctgcactcgctctgcctgcggaggaacaaggtggccattgtggtcagctcgctggac

SEQ ID NO: 9 - DNA, 72 bp, Homo sapiens
gactgggtttggaacctggagaaaatggacttgtcgggcaacgagatcgagtacatggagccccatgtgttc

SEQ ID NO: 10 - DNA, 72 bp, Homo sapiens
gagaccgtgccgcacctgcagtccctgcagctggactccaaccgcctcacctacatcgagccccggatcctc

SEQ ID NO: 11 - DNA, 75 bp, Homo sapiens
gccgcccccagcgggtgcccgcagctgtgccggtgcgaggggcggctgctgtactgcgaggcgctcaacctcacc

SEQ ID NO: 12 - DNA, 123 bp, Homo sapiens
ctgacaagcatcaccctggccgggaacctgtgggattgcgggcgcaacgtgtgtgccctagcctcgtggctcaacaacttccaggggcgctacgatggcaacttgcagtgcgccagcccggag

SEQ ID NO: 13 - DNA, 1569 bp, Homo sapiens
atggatttcctgctgctcggtctctgtctatactggctgctgaggaggccctcgggggtggtcttgtgtctgctgggggcctgctttcagatgctgcccgccgcccccagcgggtgcccgcagctgtgccggtgcgaggggcggctgctgtactgcgaggcgctcaacctcaccgaggcgccccacaacctgtccggcctgctgggcttgtccctgcgctacaacagcctctcggagctgcgcgccggccagttcacggggttaatgcagctcacgtggctctatctggatcacaatcacatctgctccgtgcagggggacgcctttcagaaactgcgccgagttaaggaactcacgctgagttccaaccagatcacccaactgcccaacaccaccttccggcccatgcccaacctgcgcagcgtggacctctcgtacaacaagctgcaggcgctcgcgcccgacctcttccacgggctgcggaagctcaccacgctgcatatgcgggccaacgccatccagtttgtgcccgtgcgcatcttccaggactgccgcagcctcaagtttctcgacatcggatacaatcagctcaagagtctggcgcgcaactctttcgccggcttgtttaagctcaccgagctgcacctcgagcacaacgacttggtcaaggtgaacttcgcccacttcccgcgcctcatctccctgcactcgctctgcctgcggaggaacaaggtggccattgtggtcagctcgctggactgggtttggaacctggagaaaatggacttgtcgggcaacgagatcgagtacatggagccccatgtgttcgagaccgtgccgcacctgcagtccctgcagctggactccaaccgcctcacctacatcgagccccggatcctcaactcttggaagtccctgacaagcatcaccctggccgggaacctgtgggattgcgggcgcaacgtgtgtgccctagcctcgtggctcaacaacttccaggggcgctacgatggcaacttgcagtgcgccagcccggagtacgcacagggcgaggacgtcctggacgccgtgtacgccttccacctgtgcgaggatggggccgagcccaccagcggccacctgctctcggccgtcaccaaccgcagtgatctggggccccctgccagctcggccaccacgctcgcggacggcggggaggggcagcacgacggcacattcgagcctgccaccgtggctcttccaggcggcgagcacgccgagaacgccgtgcagatccacaaggtggtcacgggcaccatggccctcatcttctccttcctcatcgtggtcctggtgctctacgtgtcctggaagtgtttcccagccagcctcaggcagctcagacagtgctttgtcacgcagcgcaggaagcaaaagcagaaacagaccatgcatcagatggctgccatgtctgcccaggaatactacgttgattacaaaccgaaccacattgagggagccctggtgaccatcaacgagtatggctcgtgtacctgccaccagcagcccgcgagggaatgcgaggtgtga

SEQ ID NO: 14 - protein, 24 aa, Homo sapiens
HNLSGLLGLSLRYNSLSELRAGQF

SEQ ID NO: 15 - protein, 24 aa, Homo sapiens
TGLMQLTWLYLDHNHICSVQGDAF

SEQ ID NO: 16 - protein, 24 aa, Homo sapiens
QKLRRVKELTLSSNQITQLPNTTF

SEQ ID NO: 17 - protein, 24 aa, Homo sapiens
RPMPNLRSVDLSYNKLQALAPDLF

SEQ ID NO: 18 - protein, 24 aa, Homo sapiens
HGLRKLTTLHMRANAIQFVPVRIF

SEQ ID NO: 19 - protein, 24 aa, Homo sapiens
QDCRSLKFLDIGYNQLKSLARNSF

SEQ ID NO: 20 - protein, 24 aa, Homo sapiens
AGLFKLTELHLEHNDLVKVNFAHF

SEQ ID NO: 21 - protein, 24 aa, Homo sapiens
PRLISLHSLCLRRNKVAIVVSSLD

SEQ ID NO: 22 - protein, 24 aa, Homo sapiens
DWVWNLEKMDLSGNEIEYMEPHVF

SEQ ID NO: 23 - protein, 24 aa, Homo sapiens
ETVPHLQSLQLDSNRLTYIEPRIL

SEQ ID NO: 24 - protein, 25 aa, Homo sapiens
AAPSGCPQLCRCEGRLLYCEALNLT

SEQ ID NO: 25 - protein, 41 aa, Homo sapiens
LTSITLAGNLWDCGRNVCALASWLNNFQGRYDGNLQCASPE

SEQ ID NO: 26 - protein, 522 aa, Homo sapiens [sequence omitted]

SEQ ID NO: 27 - DNA, 4104 bp, Homo sapiens
cccacgcgtccgcccacgcgtccgcccacgcgtccgcccacgcgtccgcccacgcgtccgcccacgcgtccgcccacgcgtccggtgcaagctcgcgccgcacactgcctggtggagggaaggagcccgggcgcctctcgccgctccccgcgccgccgtccgcacctccccaccgcccgccgcccgccgcccgccgcccgcaaagcatgagtgagcccgctctctgcagctgcccggggcgcgaatggcaggctgtttccgcggagtaaaaggtggcgccggtcagtggtcgtttccaatgacggacattaaccagactgtcagatcctggggagtcgcgagccccgagtttggagttttttccccccacaacgtcacagtccgaactgcagagggaaaggaaggcggcaggaaggcgaagctcgggctccggcacgtagttgggaaacttgcgggtcctagaagtcgcctccccgccttgccggccgcccttgcagccccgagccgagcagcaaagtgagacattgtgcgcctgccagatccgccggccgcggaccggggctgcctcggaaacacagaggggtcttctctcgccctgcatataattagcctgcacacaaagggagcagctgaatggaggttgtcactctctggaaaaggatttctgaccgagcgcttccaatggacattctccagtctctctggaaagattctcgctaatggatttcctgctgctcggtctctgtctatactggctgctgaggaggccctcgggggtggtcttgtgtctgctgggggcctgctttcagatgctgcccgccgcccccagcgggtgcccgcagctgtgccggtgcgaggggcggctgctgtactgcgaggcgctcaacctcaccgaggcgccccacaacctgtccggcctgctgggcttgtccctgcgctacaacagcctctcggagctgcgcgccggccagttcacggggttaatgcagctcacgtggctctatctggatcacaatcacatctgctccgtgcagggggacgcctttcagaaactgcgccgagttaaggaactcacgctgagttccaaccagatcacccaactgcccaacaccaccttccggcccatgcccaacctgcgcagcgtggacctctcgtacaacaagctgcaggcgctcgcgcccgacctcttccacgggctgcggaagctcaccacgctgcatatgcgggccaacgccatccagtttgtgcccgtgcgcatcttccaggactgccgcagcctcaagtttctcgacatcggatacaatcagctcaagagtctggcgcgcaactctttcgccggcttgtttaagctcaccgagctgcacctcgagcacaacgacttggtcaaggtgaacttcgcccacttcccgcgcctcatctccctgcactcgctctgcctgcggaggaacaaggtggccattgtggtcagctcgctggactgggtttggaacctggagaaaatggacttgtcgggcaacgagatcgagtacatggagccccatgtgttcgagaccgtgccgcacctgcagtccctgcagctggactccaaccgcctcacctacatcgagccccggatcctcaactcttggaagtccctgacaagcatcaccctggccgggaacctgtgggattgcgggcgcaacgtgtgtgccctagcctcgtggctcagcaacttccaggggcgctacgatggcaacttgcagtgcgccagcccggagtacgcacagggcgaggacgtcctggacgccgtgtacgccttccacctgtgcgaggatggggccgagcccaccagcggccacctgctctcggccgtcaccaaccgcagtgatctggggccccctgccagctcggccaccacgctcgcggacggcggggaggggcagcacgacggcacattcgagcctgccaccgtggctcttccaggcggcgagcacgccgagaacgccgtgcagatccacaaggtggtcacgggcaccatggccctcatcttctccttcctcatcgtggtcctggtgctctacgtgtcctggaagtgtttcccagccagcctcaggcagctcagacagtgctttgtcacgcagcgcaggaagcaaaagcagaaacagaccatgcatcagatggctgccatgtctgcccaggaatactacgttgattacaaaccgaaccacattgagggagccctggtgatcatcaacgagtatggctcgtgtacctgccaccagcagcccgcgagggaatgcgaggtgtgattgtcccagtggctctcaacccatgcgctaccaaatacgcctgggcagccgggacgggccggcgggcaccaggctggggtctccttgtctgtgctctgatatgctccttgactgaaactttaaggggatctctcccagagacttgacattttagctttattgtgtcttaaaaacaaaagcgaattaaaacacaacaaaaaaccccaccccacaaccttcaggacagtctatcttaaatttcatatgagaactccttcctccctttgaagatctgtccatattcaggaatctgagagtgtaaaaaaggtggccataagacagagagagaataatcgtgctttgttttatgctactcctcccaccctgcccatgattaaacatcatgtatgtagaagatcttaagtccatacgcatttcatgaagaaccattggaaagaggaatctgcaatctgggagcttaagagcaaatgatgaccatagaaagctatgttcttactttgtgtgtgtgtctgtatgtttctgcgttgtgtgtctttgtaggcaagcaaacgttgtctacacaaacgggaatttagctcacatcatttcatgcccctgtgcctctagctctggagattggtggggggaggtggggggaaacggcaggaataagggaaagtggtagttttaactaaggttttgtaacacttgaaatcttttctttctcaaattaattatctttaagcttcaagaaacttgctctgacccctctaagcaaactactaagcatttaaaagagaatctaatttttaaaggtgtagcacctttttttttattcttcccacagagggtgctaatctcattatgctgtgctatctgaaaagaacttaaggccacaattcacgtctcgtcctgggcattgtgatggattgaccctccatttgcagtaccttcccagctgattaaagttcagcagtggtattgaggtttttcgaatatttatatagaaaaaaagtcttttcacatgacaaatgacactctcacaccagtcttagccctagtagttttttaggttggaccagaggaagcaggttaaatgagacctgtcctctgctgcactcagaaaaaataggcagtccctgatgctcagatcttagccttgatattaatagttgagaccacctacccacaatgcagcctatactcccaagactacaaagttaccatcgcaaaggaaaggttattccagtaaaaggaaatagttttctcaaccatttaaaaatattcttctgaactcatcaaagtagaagagcccccaaccttttctctctgccttcaagaaggcagacatttggtatgatttagcatcaacaacacatttatgagtatatgtaagtaatcagaggggcaaatgccacttgttattcctcccaagttttccaagcaagtacacacagatctctggtaggattaggggccacttgtgtttccggcttattttagtcgacttgtcagcaagtttgatgcctagtctatctgacatggcccagtagaacagggcattgatggatcacatgagatggtagaaggaacatcatcacatacccctctcacagagaaaattatcaaagaaccagaaattatatctgttttggagcaagagtgtcataatgtttcagggtagtcaaaataaacataaattatctcctctagatgagtggcgatgttggctgatttgggtctgccattgacagaatgtcaaataaaaaggaattagctagaatatgaccattaaatgtgcttctgaaatatattttgagataggtttagaatgtca

SEQ ID NO: 28 - protein, 522 aa, Homo sapiens
MDFLLLGLCLYWLLRRPSGVVLCLLGACFQMLPAAPSGCPQLCRCEGRLLYCEALNLTEAPHNLSGLLGLSLRYNSLSELRAGQFTGLMQLTWLYLDHNHICSVQGDAFQKLRRVKELTLSSNQITQLPNTTFRPMPNLRSVDLSYNKLQALAPDLFHGLRKLTTLHMRANAIQFVPVRIFQDCRSLKFLDIGYNQLKSLARNSFAGLFKLTELHLEHNDLVKVNFAHFPRLISLHSLCLRRNKVAIVVSSLDWVWNLEKMDLSGNEIEYMEPHVFETVPHLQSLQLDSNRLTYIEPRILNSWKSLTSITLAGNLWDCGRNVCALASWLSNFQGRYDGNLQCASPEYAQGEDVLDAVYAFHLCEDGAEPTSGHLLSAVTNRSDLGPPASSATTLADGGEGQHDGTFEPATVALPGGEHAENAVQIHKVVTGTMALIFSFLIVVLVLYVSWKCFPASLRQLRQCFVTQRRKQKQKQTMHQMAAMSAQEYYVDYKPNHIEGALVIINEYGSCTCHQQPARECEV

SEQ ID NO: 29 - DNA, 17 bp, Artificial Sequence
  Description of Artificial Sequence Synthetic primer
gtcacgcagcgcaggaa

SEQ ID NO: 30 - DNA, 20 bp, Artificial Sequence
  Description of Artificial Sequence Synthetic primer
gacatggcagccatctgatg

SEQ ID NO: 31 - DNA, 18 bp, Artificial Sequence
  Description of Artificial Sequence Synthetic probe
aaagcagaaacagaccat

SEQ ID NO: 32 - protein, 4 aa, Artificial Sequence
  Description of Artificial Sequence Synthetic peptide
NLTE

SEQ ID NO: 33 - protein, 4 aa, Artificial Sequence
  Description of Artificial Sequence Synthetic peptide
NLSG

SEQ ID NO: 34 - protein, 4 aa, Artificial Sequence
  Description of Artificial Sequence Synthetic peptide
NTTF

SEQ ID NO: 35 - protein, 4 aa, Artificial Sequence
  Description of Artificial Sequence Synthetic peptide
NRSD

SEQ ID NO: 36 - protein, 15 aa, Artificial Sequence
  Description of Artificial Sequence Synthetic peptide
LSGNEIEYMEPHVFE

SEQ ID NO: 37 - protein, 15 aa, Artificial Sequence
  Description of Artificial Sequence Synthetic peptide
LQCASPEYAGGEDVL

SEQ ID NO: 38 - protein, 4 aa, Artificial Sequence
  Description of Artificial Sequence Synthetic peptide
RRPS

SEQ ID NO: 39 - protein, 4 aa, Artificial Sequence
  Description of Artificial Sequence Synthetic peptide
RKLT

SEQ ID NO: 40 - protein, 4 aa, Artificial Sequence
  Description of Artificial Sequence Synthetic peptide
SLSE

SEQ ID NO: 41 - protein, 4 aa, Artificial Sequence
  Description of Artificial Sequence Synthetic peptide
SSLD

SEQ ID NO: 42 - protein, 4 aa, Artificial Sequence
  Description of Artificial Sequence Synthetic peptide
SGNE

SEQ ID NO: 43 - protein, 4 aa, Artificial Sequence
  Description of Artificial Sequence Synthetic peptide
TYIE

SEQ ID NO: 44 - protein, 4 aa, Artificial Sequence
  Description of Artificial Sequence Synthetic peptide
TLAD

SEQ ID NO: 45 - protein, 4 aa, Artificial Sequence
  Description of Artificial Sequence Synthetic peptide
SAQE

SEQ ID NO: 46 - protein, 4 aa, Artificial Sequence
  Description of Artificial Sequence Synthetic peptide
TINE

SEQ ID NO: 47 - protein, 7 aa, Artificial Sequence
  Description of Artificial Sequence Synthetic peptide
RSVDLSY

SEQ ID NO: 48 - protein, 7 aa, Artificial Sequence
  Description of Artificial Sequence Synthetic peptide
KFLDIGY

SEQ ID NO: 49 - protein, 6 aa, Artificial Sequence
  Description of Artificial Sequence Synthetic peptide
GVVLCL

SEQ ID NO: 50 - protein, 6 aa, Artificial Sequence
  Description of Artificial Sequence Synthetic peptide
GQFTGL

SEQ ID NO: 51 - protein, 6 aa, Artificial Sequence
  Description of Artificial Sequence Synthetic peptide
GNLQCA

SEQ ID NO: 52 - protein, 6 aa, Artificial Sequence
  Description of Artificial Sequence Synthetic peptide
GAEPTS

SEQ ID NO: 53 - protein, 6 aa, Artificial Sequence
  Description of Artificial Sequence Synthetic peptide
GQHDGT

SEQ ID NO: 54 - protein, 6 aa, Artificial Sequence
  Description of Artificial Sequence Synthetic peptide
GGEHAE

SEQ ID NO: 55 - protein, 6 aa, Artificial Sequence
  Description of Artificial Sequence Synthetic peptide
GALVTI

SEQ ID NO: 56 - protein, 6 aa, Artificial Sequence
  Description of Artificial Sequence Synthetic peptide
GSCTCH

SEQ ID NO: 57 - protein, 4 aa, Artificial Sequence
  Description of Artificial Sequence Synthetic peptide
ECEV